(19) United States
(12) Patent Application Publication
(10) Pub. No.: US 2010/0093824 A1
(43) Pub. Date: Apr. 15, 2010

(54) METHODS OF TREATING VIRAL INFECTION

(76) Inventors: Judith Frydman, Stanford, CA (US); Raul Andino-Pavlovsky, San Francisco, CA (US); Ron Geller, Menlo Park, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/515,143

(22) PCT Filed: Nov. 28, 2007
(86) PCT No.: PCT/US07/24608

§ 371 Date: Dec. 17, 2009

Related U.S. Application Data
(60) Provisional application No. 60/867,742, filed on Nov. 29, 2006.

(51) Int. Cl. A61K 31/4025; A61K 31/33; A61K 31/365; A61K 31/19

(52) U.S. Cl. 514/422; 514/183; 514/475; 514/575

(57) ABSTRACT

The present invention provides methods of treating an RNA viral infection, generally involving administering an agent that reduces the activity of a host cell protein required for maturation of a viral protein, where the emergence of variant virus resistant to the agent is reduced. The present invention further provides combination therapies for viral infection, involving administration of two or more agents that reduce the activity of a host cell protein required for maturation of a viral protein.

### CROSS-REFERENCE

This application claims the benefit of U.S. Provisional Patent Application No. 60/867,742, filed Nov. 29, 2006, which application is incorporated herein by reference in its entirety.

### STATEMENT REGARDING FEDERALLY SPONSORED RESEARCH

The U.S. government may have certain rights in this invention, pursuant to grant nos. GM56433 and AI40085 awarded by the National Institutes of Health.

### BACKGROUND

RNA viruses possess the greatest capacity for rapid evolution among all organisms. Their ability to adapt stems from having the highest mutation rates in nature, combined with short generation times, and very large population sizes. In fact, RNA viruses never exist as a single species; rather, at any single time, the viral population consists of an ensemble of closely related genotypes termed “quasi-species.” This property allows RNA viruses to evolve at rates of up to a million times greater than those observed for organisms employing DNA to encode their genome. Such capacity for rapid evolution enables viruses to survive in the face of adverse conditions and successfully replicate in different hosts and changing microenvironments.

The tremendous capacity of viruses for rapid evolution has profound medical consequences as many antiviral drugs are rendered ineffective by the emergence of drug resistant viral variants. The most common antiviral strategy relies on directly inhibiting viral proteins. While leading to specific viral inhibitors, this strategy invariably results in the emergence of drug resistance as the virus can readily mutate to circumvent inhibition, even under conditions of combinatorial therapy targeting multiple viral proteins. An alternative strategy is to target host processes required for viral replication, as direct mutation of the drug target is not possible. Strikingly, this approach also results in the emergence of viral drug resistance. For instance, poliovirus replication is strongly inhibited by Brefeldin A (BFA), which targets components of the cellular secretory apparatus required for viral RNA replication. However, viral variants independent of these factors and resistant to BFA were readily isolated. Human immunodeficiency virus (HIV) can also rapidly gain resistance to an inhibitor of a cellular prolyl-peptidyl isomerase that is required for infectivity. Likewise, herpes simplex virus (HSV) can become resistant to an inhibitor of a nuclear export factor, Crm1, involved in export of HSV viral RNAs from the nucleus. In such cases, the viruses are thought to gain drug resistance by evolving new replication strategies that use alternate cellular factors or dispense with the affected function.

There is a need in the art for improved methods for treating viral infections, where the treatment methods are less likely to yield resistant variants of the virus.

### LITERATURE

Li et al. (2004) Antimicrobial Agents and Chemotherapy 48:867-872; Hung et al. (2002) J. Virol. 76:1379-1390; Hu et al. (2004) J. Virol. 78:13122-13131; Dalton et al. (2006) Virology J. 3:58; Momose et al. (2002) J. Biol. Chem. 277:45306; Valenzuela-Fernandez et al. (2005) Mol. Biol. Cell 16:5445; Okamoto et al. (2006) EMBO J. 25:5015; WO 2007/058384; Ju and Seeger (1996) Proc. Natl. Acad. Sci. USA 93:1060; Okamoto et al. (2006) EMBO J. 25:5015; Braaten et al. (1996) J. Virol. 70:5170; Murata et al. (2001) J. Virol. 75:1039; Crotty et al. (2004) J. Virol. 78:3378.

### SUMMARY OF THE INVENTION

The present invention provides methods of treating an RNA viral infection, generally involving administering an agent that reduces the activity of a host cell protein required for maturation of a viral protein, where the emergence of variant virus resistant to the agent is reduced. The present invention further provides combination therapies for viral infection, involving administration of two or more agents that reduce the activity of a host cell protein required for maturation of a viral protein.

### BRIEF DESCRIPTION OF THE DRAWINGS

FIGS. 1A-E depict the effect of the Hsp90 inhibitor geldanamycin (GA) on picornavirus replication in cultured cells.

FIGS. 2A-F depict the effect of inhibition of Hsp90 on production of mature capsid proteins.

FIGS. 3A-F depict association of Hsp90 with the capsid precursor P1 and its requirement for processing to mature capsid proteins.

FIGS. 4A and 4B depict sensitivity of poliovirus to GA over numerous passages.

FIGS. 5A-C depict inhibition of viral replication by GA, without appearance of drug-resistant variants.

FIG. 6 depicts inhibition of viral replication by 17-AAG in poliovirus-infected animals.

FIG. 7 depicts inhibition of virus production by GA, when GA is added after viral entry and uncoating have occurred.

FIGS. 8A and 8B depict GA inhibition of rhinovirus P1 processing.

FIG. 9 depicts Hsp90 binding to viral protein in poliovirus-infected cells.

FIG. 10 depicts the effect of an HDAC inhibitor (TSA) and an Hsp90 inhibitor (GA) on virus production in virus-infected cells.

FIG. 11 depicts the effect of 17-AAG on Respiratory Syncytial Virus production in cultured cells.

FIG. 12 depicts the effect of Hsp90 inhibition on L protein, a Respiratory Syncytial virus polymerase.

FIG. 13 depicts the effect of 17-AAG on Influenza A virus replication in cultured cells.

FIG. 14 depicts the effect of 17-AAG on Yellow Fever Virus replication in cultured cells.

### DEFINITIONS

As used herein, the term “a host cell protein that is required for maturation of one or more proteins encoded by an RNA virus” refers to a protein that carries out one or more of: i) folding; ii) assembly; and iii) intracellular localization, of one or more proteins encoded by an RNA virus. A host cell protein that is required for maturation of one or more proteins encoded by an RNA virus has an effect on maturation of the virally-encoded protein, and thereby affects a level and/or an activity of the protein.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” and “patient,” used interchangeably herein, refer to a mammal, including, but not limited to, murines, simians, humans, mammalian farm animals, mammalian sport animals, and mammalian pets.

As used herein, the term “flavivirus” includes any member of the family Flaviviridae, including, but not limited to, Dengue virus, including Dengue virus 1, Dengue virus 2, Dengue virus 3, Dengue virus 4 (see, e.g., GenBank Accession Nos. M23027, M19197, A34774, and M14931); Yellow Fever Virus; West Nile Virus; Japanese Encephalitis Virus; St. Louis Encephalitis Virus; Bovine Viral Diarrhea Virus (BVDV); and Hepatitis C Virus (HCV); and any serotype, strain, genotype, subtype, quasispecies, or isolate of any of the foregoing. Where the flavivirus is HCV, the HCV is any of a number of genotypes, subtypes, or quasispecies, including, e.g., genotype 1, including 1a and 1b, 2, 3, 4, 6, etc. and subtypes (e.g., 2a, 2b, 3a, 4a, 4c, etc.), and quasispecies.

The term “isolated compound” means a compound which has been substantially separated from, or enriched relative to, other compounds with which it occurs in nature. Isolated compounds are typically at least about 80%, at least about 90% pure, at least about 98% pure, at least about 99%, or greater than 99%, pure, by weight. The present invention relating to active compounds is meant to comprehend diastereomers as well as their racemic and resolved, enantiomerically pure forms and pharmaceutically acceptable salts thereof.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the active agents of the present invention depend on the particular compound and the effect to be achieved, and the pharmacodynamics associated with each compound in the host.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectable system); and a single subcutaneous injection followed by installation of a continuous delivery system.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and generally free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal and the like. In some embodiments the composition is suitable for administration by an oral route of administration. In some embodiments the composition is suitable for administration by an inhalation route of administration. In some embodiments the composition is suitable for administration by a transdermal route, e.g., using a penetration enhancer. In other embodiments, the pharmaceutical compositions are suitable for administration by a route other than transdermal administration.

As used herein, “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a heat shock protein inhibitor” includes a plurality of such inhibitors and reference to “the HDAC inhibitor” includes reference to one or more HDAC inhibitors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides methods of treating an RNA viral infection, generally involving administering an agent that reduces the activity of a host cell protein required for maturation of a viral protein, where the emergence of variant virus resistant to the agent is reduced. The present invention further provides combination therapies for viral infection, involving administration of two or more agents that reduce the activity of a host cell protein required for maturation of a viral protein.

### Methods of Treating RNA Viral Infections

The present invention provides methods of treating a virus infection, and methods of reducing viral load, or reducing the risk that an individual will develop a viral infection, or reducing the time to viral clearance, or reducing morbidity or mortality in the clinical outcomes, in patients suffering from an RNA virus infection. The methods generally involve administering to an individual in need thereof an effective amount of an active agent that reduces the activity of a host cell protein that is required for maturation of a viral protein. For example, in some embodiments, the methods generally involve administering to an individual in need thereof an effective amount of an active agent that inhibits a heat shock protein or chaperone that facilitates maturation of one or more viral proteins. The effect of the active agent is not a direct effect on replication or translation; instead, the active agent acts directly on a host protein, e.g., a heat shock protein or a chaperone protein, which heat shock protein or chaperone protein facilitates maturation of one or more viral proteins.

The methods are effective to treat an RNA viral infection, without substantial emergence of variant viruses that are resistant to the agent. In some embodiments, the methods are effective to treat an infection caused by a positive-strand RNA virus. In other embodiments, the methods are effective to treat an infection caused by a negative-strand RNA virus.

In some embodiments, the viral infection is caused by a virus of family Flaviviridae. In some embodiments, the virus of family Flaviviridae is selected from Yellow Fever Virus, West Nile virus, dengue fever virus, and Hepatitis C Virus. In other embodiments, the viral infection is caused by a virus of family Picornaviridae, e.g., poliovirus, rhinovirus, coxsackievirus, etc. In other embodiments, the viral infection is caused by a member of Orthomyxoviridae, e.g., an influenza virus. In other embodiments, the viral infection is caused by a member of Retroviridae, e.g., a lentivirus. In other embodiments, the viral infection is caused by a member of Paramyxoviridae, e.g., respiratory syncytial virus, a human parainfluenza virus, rubulavirus (e.g., mumps virus), measles virus, and human metapneumovirus. In other embodiments, the viral infection is caused by a member of Bunyaviridae, e.g., hantavirus. In other embodiments, the viral infection is caused by a member of Reoviridae, e.g., a rotavirus. In some embodiments, the virus is one that infects humans. In other embodiments, the virus is one that infects a non-human mammal, e.g., the virus is one that infects a mammalian livestock animal, e.g., a cow, a horse, a pig, a goat, a sheep, etc.

Suitable active agents include agents that reduce the activity of a host cell protein that is required for maturation of a viral protein. For example, a suitable active agent for inhibiting a picornaviral infection is an agent that reduces the activity of a host protein in effecting maturation of picornavirus capsid protein P1. Suitable active agents include agents that reduce the activity of a heat shock protein. Agents that reduce the activity of a heat shock protein include agents that inhibit Hsp90 directly, e.g., inhibit the activity of Hsp90 that provides for maturation of a viral protein. Agents that inhibit Hsp90 include agents that bind with high affinity to the N-terminus pocket of Hsp90, thereby destabilizing substrates that normally interact with Hsp90. Agents that reduce the activity of Hsp90 in effecting maturation of a viral protein include agents that inhibit deacetylation of Hsp90. Suitable active agents further include agents that reduce the activity of an Hsp-dependent host cell protein that is required for viral protein maturation.

The term “Hsp90 protein” refers to a polypeptide that has at least about 70%, at least about 75%, at least about 80%, at least about 85%, at least about 90%, at least about 95%, at least about 98%, or at least about 99% amino acid sequence identity to the amino acid sequence presented in GenBank Accession No. NP_{—}031381, and set forth in SEQ ID NO:1, and that functions in the maturation of one or more viral proteins. An Hsp90 protein can have a molecular weight of about 90 kDa. In some embodiments, an Hsp90 protein functions in the maturation of a viral capsid protein.

In some embodiments, a suitable agent for use in a subject method is an agent that, when administered in one or more doses, reduces viral load in an individual by at least about 10%, at least about 20%, at least about 30%, at least about 40%, at least about 50%, at least about 60%, at least about 70%, at least about 80%, at least about 90%, at least about 95%, or more, compared to the viral load in an untreated individual. For example, a suitable agent for use in a subject method is an agent that, when administered in one or more doses, reduces viral load in an individual by at least about 10%, at least about 20%, at least about 30%, at least about 40%, at least about 50%, at least about 60%, at least about 70%, at least about 80%, at least about 90%, at least about 95%, or more, when measured at a time point following the beginning of a therapeutic regimen, e.g., when measured from about 1 day to about 14 days, e.g., from about 1 day to 2 days, from 2 days to 4 days, or from 4 days to 7 days after the start of a therapeutic regimen with the agent.

In some embodiments, a suitable agent for use in a subject method is an agent that, when administered in one or more doses, reduces viral load in an individual, as described above, and which does not give rise to substantial numbers of variant viruses that are resistant to the agent. For example, a suitable agent for use in a subject method is an agent that, when administered in one or more doses, reduces viral load in an individual, as described above, where viral variants that are resistant to the agent, if present in any detectable numbers, are present in an amount of less than about 10² viral genomes/mL serum, less than about 10 viral genomes/mL serum, or less than about 1 viral genome/mL serum. For example, a suitable agent for use in a subject method is an agent that, when administered in one or more doses, reduces viral load in an individual, as described above, where viral variants that are resistant to the agent, if present in any detectable numbers, are present in an amount of less than about 10² viral genomes/mL serum, less than about 10 viral genomes/mL serum, or less than about 1 viral genome/mL serum, 1 day, 2 days, 3 days, 4 days, 5 days, 6 days, 7 days, or from about 7 days to about 30 days, following the start of a therapeutic regimen with the agent. In some embodiments, a suitable agent for use in a subject method is an agent that, when administered in one or more doses, reduces viral load in an individual, as described above, where viral variants that are resistant to the agent are undetectable. In some embodiments, a suitable agent for use in a subject method is an agent that, when administered in one or more doses, reduces viral load in an individual, as described above, where viral variants that are resistant to the agent are undetectable 1 day, 2 days, 3 days, 4 days, 5 days, 6 days, 7 days, or from about 7 days to about 30 days, following the start of a therapeutic regimen with the agent.

In some embodiments, an effective amount of an active agent is an amount that reduces the risk that a person who has been exposed to an RNA virus, but who has not yet exhibited symptoms of infection by the RNA virus, will develop disease symptoms resulting from infection by the RNA virus.

In some embodiments, an effective amount of an active agent (e.g., an Hsp90 inhibitor) is an amount that that reduces the time to viral clearance, by at least about 10%, at least about 20%, at least about 25%, at least about 30%, at least about 35%, at least about 40%, at least about 50%, at least about 60%, at least about 70%, at least about 80%, at least about 90%, or more, compared to the time to viral clearance in the absence of treatment with the agent.

In some embodiments, an effective amount of an active agent (e.g., an Hsp90 inhibitor) is an amount that reduces morbidity or mortality due to a virus infection by at least about 10%, at least about 20%, at least about 25%, at least about 30%, at least about 35%, at least about 40%, at least about 50%, at least about 60%, at least about 70%, at least about 80%, at least about 90%, or more, compared to the morbidity or mortality in the absence of treatment with the agent.

Whether a subject treatment method is effective in reducing viral load, reducing time to viral clearance, or reducing morbidity or mortality due to a virus infection is readily determined by those skilled in the art. Viral load is readily measured by measuring the titer or level of virus in serum. The number of virus in the serum can be determined using any known assay, including, e.g., a quantitative polymerase chain reaction assay using oligonucleotide primers specific for the virus being assayed. Whether morbidity is reduced can be determined by measuring any symptom associated with a virus infection, including, e.g., fever, respiratory symptoms (e.g., cough, ease or difficulty of breathing, and the like).

In some embodiments, the present invention provides methods of reducing viral load, and/or reducing the time to viral clearance, and/or reducing morbidity or mortality in an individual who has not been infected with a virus, and who has been exposed to a virus. In some of these embodiments, the methods involve administering an effective amount of an active agent (e.g., an Hsp90 inhibitor) within 48 hours of exposure to the virus. In other embodiments, the methods involve administering an active agent (e.g., an Hsp90 inhibitor) more than 48 hours after exposure to the virus, e.g., from 72 hours to about 35 days, e.g., 72 hours, 4 days, 5 days, 6 days, or 7 days after exposure, or from about 7 days to about 10 days, from about 10 days to about 14 days, from about 14 days to about 17 days, from about 17 days to about 21 days, from about 21 days to about 25 days, from about 25 days to about 30 days, or from about 30 days to about 35 days after exposure to the virus.

A therapeutic regimen comprises administering to an individual in need thereof a therapeutically effective amount an active agent that inhibits a heat shock protein or chaperone that facilitates maturation of one or more viral proteins. In some embodiments, multiple doses of an active agent are administered. The frequency of administration of an active agent can vary depending on any of a variety of factors, e.g., severity of the symptoms, etc. For example, in some embodiments, an active agent is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an active agent, e.g., the period of time over which an active agent is administered, can vary, depending on any of a variety of factors, e.g., patient response, etc. For example, an active agent can be administered over a period of time ranging from about one day to about 2 days, from about 2 days to about 4 days, from about 4 days to about one week, from about two weeks to about four weeks, from about one month to about two months, from about two months to about four months, or longer than four months.

### Picornaviridae Infection

The present invention provides methods for treating a Picornaviridae infection (also referred to as a “picornaviral infection”), e.g., an infection with a member of the Picornaviridae family. In general, a subject method for treating a picornaviral infection comprises administering an effective amount of an active agent (e.g., an Hsp90 inhibitor), as described above. The picornavirus infection may be caused by any virus of the family Picornaviridae. Representative family members include human rhinoviruses, polioviruses, enteroviruses including coxsackieviruses and echoviruses, hepatovirus, cardioviruses, apthovirus, hepatitis A and other picornaviruses not yet assigned to a particular genus, including one or more of the serotypes of these viruses.

Whether an active agent (e.g., an Hsp90 inhibitor) is effective to treat a picornavirus infection can be determined using any of a variety of assays. For example, an animal model of a picornavirus infection can be used to determine whether a given active agent is effective to reduce viral load. In a human subject, efficacy of an active agent can be determined by measuring viral load and/or measuring one or more symptoms of a picornaviral infection.

### Flaviviridae Infection

The present invention provides methods for treating a Flaviviridae infection (also referred to as a “flavirirus infection”), e.g., an infection with a member of the Flaviviridae family. In general, a subject method for treating a flavivirus infection comprises administering an effective amount of an active agent (e.g., an Hsp90 inhibitor), as described above.

In some embodiments, a subject method provides for treatment of a Dengue virus infection. In other embodiments, a subject method provides for treatment of a West Nile Virus infection. In other embodiments, a subject method provides for treatment of a Yellow Fever Virus infection. In other embodiments, a subject method provides for treatment of an HCV infection. In some embodiments, a subject method provides for treatment of an HCV infection, wherein the HCV is a drug-resistant HCV, e.g., the HCV is resistant to treatment with a drug other than an active agent described herein, e.g., the HCV is resistant to treatment with a drug other than an Hsp90 inhibitor.

Whether an active agent (e.g., an Hsp90 inhibitor) is effective to treat a flavivirus infection can be determined using any of a variety of assays. For example, an animal model of a flavivirus infection can be used to determine whether a given active agent is effective to reduce viral load. In a human subject, efficacy of an active agent can be determined by measuring viral load and/or measuring one or more symptoms of a flavivirus infection.

Whether an active agent (e.g., an Hsp90 inhibitor) is effective to treat an HCV infection can be determined using, e.g., an assay that measures HCV viral load. Viral load can be measured by measuring the titer or level of virus in serum. These methods include, but are not limited to, a quantitative polymerase chain reaction (PCR) and a branched DNA (bDNA) test. Quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Also of interest is a nucleic acid test (NAT), developed by Gen-Probe Inc. (San Diego) and Chiron Corporation, and sold by Chiron Corporation under the trade name Procleix®, which NAT simultaneously tests for the presence of HIV-1 and HCV. See, e.g., Vargo et al. (2002) Transfusion 42:876-885.

### Orthomyxoviridae Virus Infection

The present invention provides methods for treating an Orthomyxoviridae virus infection, e.g., an infection with a member of the family Orthomyxoviridae. In general, a subject method for treating an Orthomyxoviridae virus infection comprises administering an effective amount of an active agent (e.g., an Hsp90 inhibitor), as described above. In some embodiments, a subject method provides for treating an influenza virus infection. A subject method is suitable for treating an infection caused by any of the three types of influenza viruses: A, B, and C. A subject method is suitable for treating an infection caused by any of a variety of subtypes of influenza A virus, e.g., influenza virus of any of a variety of combinations of hemagglutinin (HA) and neuraminidase (NA) variants. Subtypes of influenza A virus that can be treated using a subject method include H1N1, H1N2, and H3N2 subtypes. Avian influenza A virus infections that can be treated with a subject method include infections with an avian influenza A virus of any one of the subtypes H5 and H7, including H5N1, H7N7, H9N2, H7N2, and H7N3 viruses. A subject method is suitable for treating an infection caused by any strain of an influenza A subtype or an influenza B virus. An infection caused by any subtype of influenza A H5, influenza A H7, and influenza A H9 can be treated using a subject method.

Whether an active agent (e.g., an Hsp90 inhibitor) is effective to treat an influenza virus infection can be determined using any of a variety of assays. For example, an animal model of an influenza virus infection can be used to determine whether a given active agent is effective to reduce viral load. In a human subject, efficacy of an active agent can be determined by measuring viral load and/or measuring one or more symptoms of an influenza virus infection.

### Paramyxoviridae Infection

The present invention provides methods for treating a Paramyxoviridae infection (also referred to as a paramyxovirus infection), e.g., an infection with a member of the family Paramyxoviridae. In general, a subject method for treating a Paramyxoviridae infection comprises administering an effective amount of an active agent (e.g., an Hsp90 inhibitor), as described above.

In some embodiments, a subject method provides for treatment of a respiratory syncytial virus (RSV) infection. RSV is the most common cause of bronchiolitis and pneumonia among infants and children under 1 year of age. In some embodiments, a subject method comprises administering an effective amount of an active agent, as described above, to an individual having an RSV infection, wherein the individual is less than 1 year of age, from about 1 year of age to about 2 years of age, from about 2 years of age to about 3 years of age, from about 3 years of age to about 4 years of age, from about 4 years of age to about 5, from about 5 years of age to about 6 years of age, or older than 6 years of age. In some embodiments, an active agent that reduces the activity of a host cell protein that is required for maturation of one or more proteins encoded by an RSV is administered in combination therapy with at least one additional therapeutic agent. For example, in some embodiments, an active agent that reduces the activity of a host cell protein that is required for maturation of one or more proteins encoded by an RSV is administered in combination therapy with ribavirin. In other embodiments, an active agent that reduces the activity of a host cell protein that is required for maturation of one or more proteins encoded by an RSV is administered in combination therapy with an HDAC inhibitor.

### Hsp90 Inhibitors

Any of a variety of Hsp90 inhibitors can be used in a subject method. Hsp90 inhibitors that are suitable for use in a subject method include the Hsp90 inhibitors described in U.S. Pat. Nos. 7,129,244; 4,261,989; 5,387,584; 5,932,566; 6,872,715; 6,887,993; 6,875,863; 6,855,705; 6,635,662; 6,316,491; 6,239,168; 6,747,055; and 6,890,917. Hsp90 inhibitors that are suitable for use in a subject method include the Hsp90 inhibitors described in U.S. Patent Publication Nos. 2006/0014730; 2006/0019941; 2006/0019939; and 2006/0014731. In some embodiments, a suitable Hsp90 inhibitor is a compound that is an ATP competitive inhibitor of Hsp90. ATP competitive inhibitors of Hsp90 include, e.g., radicicol and derivatives of radicicol; geldanamycin and derivative of geldanamycin; resorcinylic pyrazol/isoxazole amide analogues of Hsp90 inhibitors; purine-based Hsp90 inhibitors; and the like.

In some embodiments, an Hsp90 inhibitor is a compound of Formula I:

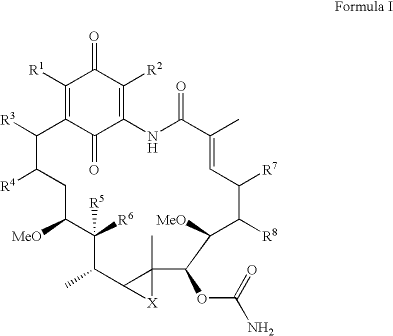

where the substituents are as described in U.S. Pat. No. 6,872,715.

In some embodiments, the agent is 17-Allylamino-17-demethoxygeldanamycin (17-AAG). 17-AAG has the following structure:

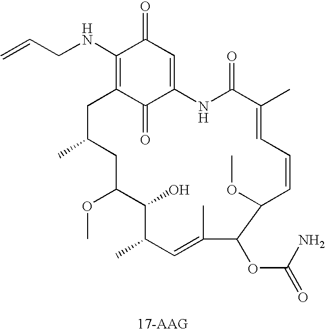

In some embodiments, the agent is 17-(Dimethylaminoethylamino)-17-demethoxygeldanamycin (17-DMAG). 17-DMAG has the following structure:

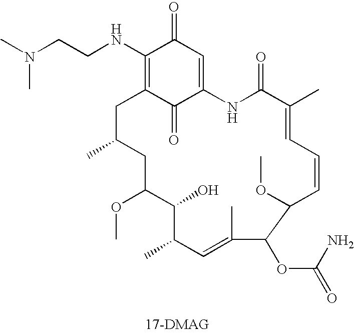

In some embodiments, the agent is 17-[2-(Pyrrolidin-1-yl)ethyl]amino-17-demethoxygeldanamycin (17-AEP-GA). 17-AEP-GA has the following structure:

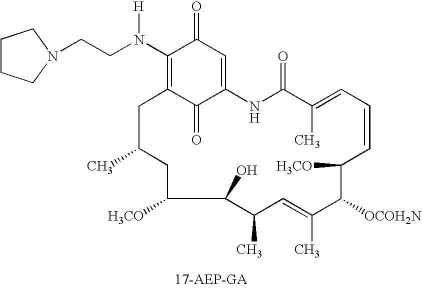

In some embodiments, the agent is 17-(Dimethylaminopropylamino)-17-demethoxygeldanamycin (17-DMAP-GA). 17-DMAP-GA has the following structure:

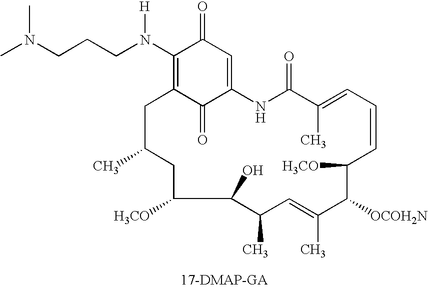

In some embodiments, the agent is an 11-O-methyl derivative of geldanamycin, e.g., a compound as described in one or more of U.S. Pat. Nos. 6,887,993, 6,875,863, 6,870,049, and 6,855,705. For example, in some embodiments, the agent is a compound of Formula II:

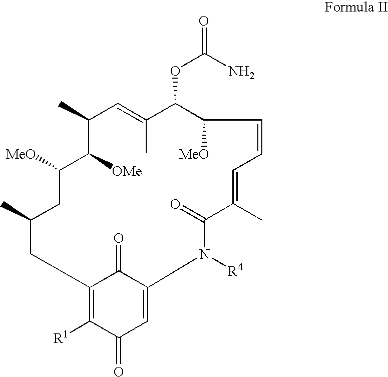

where the substituents are as described in U.S. Pat. No. 6,887,993.

In some embodiments, the agent is a hydroquinone form of 17-AAG, e.g., the agent is a compound known as IPI-504 and having the structural formula:

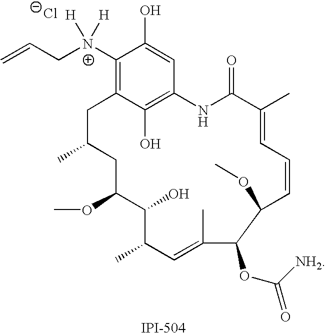

In other embodiments, an agent is a compound of Formula III:

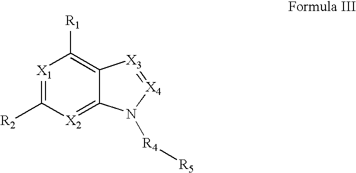

where the substituents are as described in U.S. Pat. No. 7,129,244.

In some embodiments, the agent is a pharmaceutically acceptable salt of any of the aforementioned agents, a pro-drug of any of the aforementioned agents, or a metabolite of any of the aforementioned agents.

In some embodiments, the agent is radicicol, or a derivative of radicicol, where exemplary radicicol derivatives include KF58333 (E-isomer), cycloproparadicicol, radester, pochonin D, and B-zearalenol. In some embodiments, the agent is an Hsp90 inhibitor known as radicicol and having a structural formula as shown below:

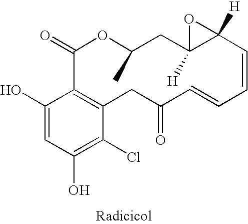

In some embodiments, the agent is an Hsp90 inhibitor known as KF58333 (E-isomer) and having a structural formula as shown below:

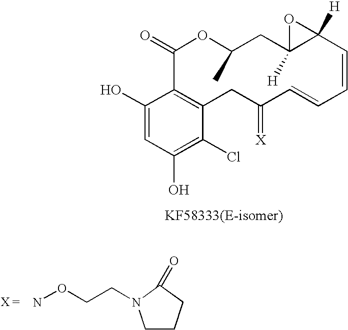

In some embodiments, the agent is an Hsp90 inhibitor known as cycloproparadicicol, and having a structural formula as shown below:

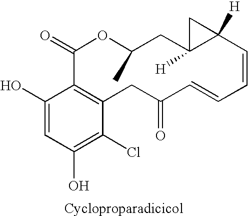

In some embodiments, the agent is an Hsp90 inhibitor known as radester, and having a structural formula as shown below:

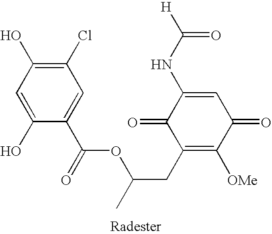

In some embodiments, the agent is an Hsp90 inhibitor known as pochonin D, and having a structural formula as shown below:

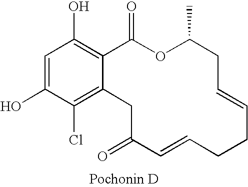

In some embodiments, the agent is an Hsp90 inhibitor known as B-zearalenol, and having a structural formula as shown below:

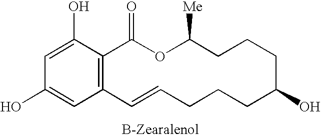

In some embodiments, the agent is a resorcinol analog (e.g., a resocinylic pyrazole/isoxazole amide analog), e.g., CCT018159, CCT012937, and CCT0130024. In some embodiments, the agent is an Hsp90 inhibitor known as CCT018159, and having a structural formula as shown below:

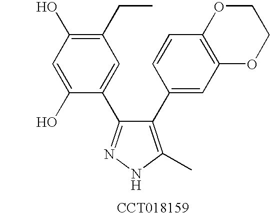

In some embodiments, the agent is an Hsp90 inhibitor known as CCT012937, and having a structural formula as shown below:

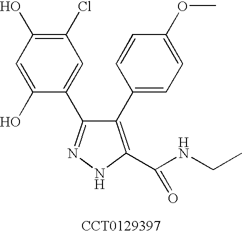

In some embodiments, the agent is an Hsp90 inhibitor known as CCT0130024, and having a structural formula as shown below:

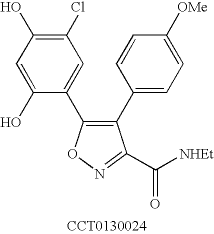

In some embodiments, the agent is a purine-based compound, e.g., a compound such as PU3, PU24FC1, and PU-H58. For example, in some embodiments, the agent is an Hsp90 inhibitor known as PU3, and having a structural formula as shown below:

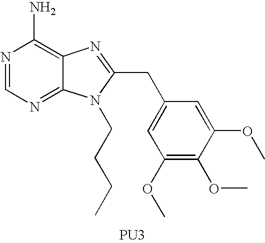

In some embodiments, the agent is an Hsp90 inhibitor known as PU24FC1, and having a structural formula as shown below:

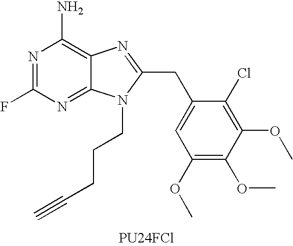

In some embodiments, the agent is an Hsp90 inhibitor known as PU-H58, and having a structural formula as shown below:

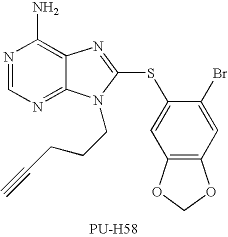

Other suitable Hsp90 inhibitors include, e.g, an antibody inhibitor, e.g., Mycograb® human recombinant antibody to Hsp90; celastrol; gedunin; agents that affect post-translation modification of Hsp90, e.g., agents that affect acetylation or phosphorylation of Hsp90, e.g., LAQ824, FK228, and the like (see, e.g., Calderwood et al., eds., Heat Shock Proteins in Cancer (2007) Springer, pages 295-329).

### Combination Therapies

In some embodiments, a subject method for treating a viral infection comprises administering a combined effective amount of two or more agents that reduce the activity of a host cell protein that is required for maturation of a viral protein. In other embodiments, a subject method for treating a viral infection comprises administering a combined effective amount of an agent that reduces the activity of a host cell protein required for maturation of a viral protein; and at least a second anti-viral agent other than an agent that reduces the activity of a host cell protein required for maturation of a viral protein.

### Combination Therapy: an Hsp Inhibitor and an HDAC Inhibitor

In some embodiments, a subject method for treating a viral infection comprises administering a combined effective amount of an Hsp90 inhibitor and an inhibitor of a histone deacetylase (HDAC). A suitable HDAC inhibitor is one that inhibits deacetylation of Hsp90. In some embodiment, a suitable HDAC inhibitor is an agent that inhibits HDAC enzymatic activity of one or more members of Class I HDACs, e.g., agents that inhibit one or more of HDAC1, HDAC2, HDAC6, and HDAC8. In other embodiments, a suitable HDAC inhibitor is a selective inhibitor of HDAC6.

In some embodiments, an Hsp90 inhibitor and an HDAC inhibitor are administered concomitantly and in the same formulation. In other embodiments, an Hsp90 inhibitor and an HDAC inhibitor are administered concomitantly and in separate formulations. In some embodiments, an Hsp90 inhibitor and an HDAC inhibitor are co-administered, e.g., are administered within about 8 hours, within about 6 hours, within about 4 hours, within about 2 hours, within about 1 hour, within about 30 minutes, within about 15 minutes, or within about 5 minutes of one another.

In some embodiments, a subject method comprises co-administering an HDAC inhibitor and an Hsp90 inhibitor, where the amount of the Hsp90 inhibitor that is administered is less than an amount of the Hsp90 inhibitor that, if administered in monotherapy for the viral infection, would be required to achieve the same reduction in viral load. In some embodiments, a subject method comprises co-administering an HDAC inhibitor and at least about 5% less, at least about 10% less, at least about 15% less, at least about 20% less, at least about 25% less, at least about 30% less, at least about 35% less, at least about 40% less, at least about 45% less, or at least about 50% less, or more than 50% less, of the amount of the Hsp90 inhibitor that, if administered in monotherapy for the viral infection, would be required to achieve the same reduction in viral load.

In some embodiments, a suitable HDAC inhibitor is a compound as described in one or more of WO 01/38322; WO 02/22577; U.S. Pat. No. 7,135,493; and U.S. Pat. No. 6,897,220.

Specific non-limiting examples of HDAC inhibitors suitable for use in the methods of the present invention are: A) Hydroxamic acid derivatives such as suberoylanilide hydroxamic acid (SAHA), pyroxamide (suberoyl-3-aminopyridineamide hydroxyamic acid), m-carboxycinnamic acid bis-hydroxamide, Trichostatin A (TSA), Trichostatin C, Salicylihydroxamic Acid (SBHA), Azelaic Bishydroxamic Acid (ABHA), Azelaic-1-Hydroxamate-9-Anilide (AAHA), 6-(3-Chlorophenylureido) carpoic Hydroxamic Acid (3Cl-UCHA), Oxamflatin, A-161906, Scriptaid, PXD-101, LAQ-824, NVP-LAQ-824 (Atadja et al., Cancer Research 64: 689-695 (2004), CHAP, MW2796, and MW2996; B) Cyclic tetrapeptides such as Trapoxin A, FR901228 (FK 228, Depsipeptide), FR225497, Apicidin, CHAP, HC-Toxin, WF27082, and Chlamydocin; C) Short Chain Fatty Acids (SCFAs) such as Sodium Butyrate, Isovalerate, Valerate, 4 Phenylbutyrate (4-PBA), Phenylbutyrate (PB), Propionate, Butyramide, Isobutyramide, Phenylacetate, 3-Bromopropionate, Tributyrin, Valproic acid and Valproate; D) Benzamide derivatives such as CI-994, MS-27-275 (MS-275) and a 3′-amino derivative of MS-27-275; E) Electrophilic ketone derivatives such as a trifluoromethyl ketone and an α-keto amide such as an N-methyl-a-ketoamide; F) Depudecin; G) porphyrin derivatives such as Trapoxin B; H) ketones such as 2-amino-8-oxo-9,10-epoxy-decanoyl; I) propenamides such as 3-(4-aroyl-1 H-pyrrol-2-yl)-N-hydroxy-2-propenamide.

In some embodiments, a suitable HDAC inhibitor is a compound of the formula:

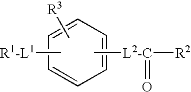

where the substituents are as described in U.S. Pat. No. 7,135,493.

In other embodiments, a suitable HDAC inhibitor is a compound of any one of the following formulas:

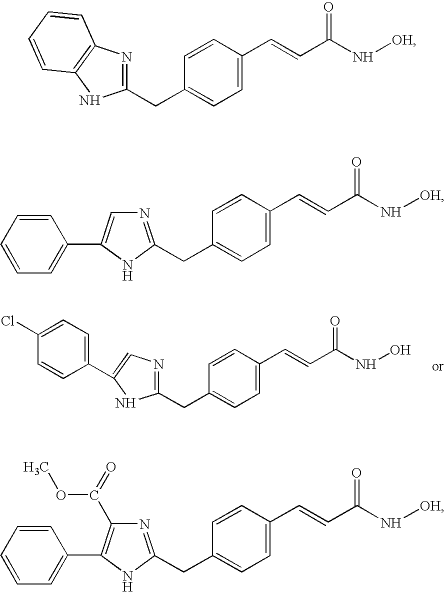

as described in U.S. Pat. No. 7,135,493.

In other embodiments, a suitable agent is a compound of the formula:

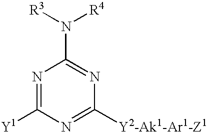

where the substituents are as described in U.S. Pat. No. 6,897,220.

For example, in some embodiments, a suitable agent is a compound of the formula:

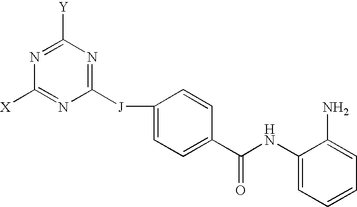

where the substituents are as described in U.S. Pat. No. 6,897,220.
Combination Therapy with a Second Anti-Viral Agent

In some embodiments, a subject method for treating a viral infection comprises administering a combined effective amount of an agent that reduces the activity of a host cell protein required for maturation of a viral protein; and at least a second anti-viral agent other than an agent that reduces the activity of a host cell protein required for maturation of a viral protein.

For example, where the infection is caused by an influenza virus, a subject method can comprise administering a combined effective amount of an agent that reduces the activity of a host cell protein required for maturation of a viral protein; and an anti-viral agent selected from amantadine, rimantadine, zanamivir, and oseltamivir. Thus, in some embodiments, a subject method comprises administering an effective amounts of: i) an agent (e.g., an Hsp90 inhibitor) that reduces the activity of a host cell protein required for maturation of a viral protein; and ii) an anti-viral agent selected from amantadine, rimantadine, zanamivir, and oseltamivir.

As another example, where the infection is caused by an HCV, a subject method can comprise administering a combined effective amount of an agent that reduces the activity of a host cell protein required for maturation of a viral protein; and an anti-viral agent selected from an NS3 inhibitor and an NS5B inhibitor. In some embodiments, a subject method comprises administering an effective amounts of: i) an agent (e.g., an Hsp90 inhibitor) that reduces the activity of a host cell protein required for maturation of a viral protein; and ii) an NS3 inhibitor. In other embodiments, a subject method comprises administering effective amounts of: i) an agent (e.g., an Hsp90 inhibitor) that reduces the activity of a host cell protein required for maturation of a viral protein; and ii) an NS5B inhibitor.

HCV non-structural protein-3 (NS3) inhibitors include, but are not limited to, a tri-peptide as disclosed in U.S. Pat. Nos. 6,642,204, 6,534,523, 6,420,380, 6,410,531, 6,329,417, 6,329,379, and 6,323,180 (Boehringer-Ingelheim); a compound as disclosed in U.S. Pat. No. 6,143,715 (Boehringer-Ingelheim); a macrocyclic compound as disclosed in U.S. Pat. No. 6,608,027 (Boehringer-Ingelheim); an NS3 inhibitor as disclosed in U.S. Pat. Nos. 6,617,309, 6,608,067, and 6,265,380 (Vertex Pharmaceuticals); an azapeptide compound as disclosed in U.S. Pat. No. 6,624,290 (Schering); a compound as disclosed in U.S. Pat. No. 5,990,276 (Schering); a compound as disclosed in Pause et al. (2003) J. Biol. Chem. 278:20374-20380; NS3 inhibitor BILN 2061 (Boehringer-Ingelheim; Lamarre et al. (2002) Hepatology 36:301A; and Lamarre et al. (Oct. 26, 2003) Nature doi:10.1038/nature02099); NS3 inhibitor VX-950 (Vertex Pharmaceuticals; Kwong et al. (Oct. 24-28, 2003) 54^{th} Ann. Meeting AASLD); NS3 inhibitor SCH6 (Abib et al. (Oct. 24-28, 2003) Abstract 137. Program and Abstracts of the 54^{th} Annual Meeting of the American Association for the Study of Liver Diseases (AASLD). Oct. 24-28, 2003. Boston, Mass.); any of the NS3 protease inhibitors disclosed in WO 99/07733, WO 99/07734, WO 00/09558, WO 00/09543, WO 00/59929 or WO 02/060926 (e.g., compounds 2, 3, 5, 6, 8, 10, 11, 18, 19, 29, 30, 31, 32, 33, 37, 38, 55, 59, 71, 91, 103, 104, 105, 112, 113, 114, 115, 116, 120, 122, 123, 124, 125, 126 and 127 disclosed in the table of pages 224-226 in WO 02/060926); an NS3 protease inhibitor as disclosed in any one of U.S. Patent Publication Nos. 2003019067, 20030187018, and 20030186895; and the like.

Suitable HCV non-structural protein-5 (NS5; RNA-dependent RNA polymerase) inhibitors include, but are not limited to, a compound as disclosed in U.S. Pat. No. 6,479,508 (Boehringer-Ingelheim); a compound as disclosed in any of International Patent Application Nos. PCT/CA02/01127, PCT/CA02/01128, and PCT/CA02/01129, all filed on Jul. 18, 2002 by Boehringer Ingelheim; a compound as disclosed in U.S. Pat. No. 6,440,985 (ViroPharma); a compound as disclosed in WO 01/47883, e.g., JTK-003 (Japan Tobacco); a dinucleotide analog as disclosed in Zhong et al. (2003) Antimicrob. Agents Chemother. 47:2674-2681; a benzothiadiazine compound as disclosed in Dhanak et al. (2002) J. Biol Chem. 277(41):38322-7; an NS5B inhibitor as disclosed in WO 02/100846 A1 or WO 02/100851 A2 (both Shire); an NS5B inhibitor as disclosed in WO 01/85172 A1 or WO 02/098424 A1 (both Glaxo SmithKline); an NS5B inhibitor as disclosed in WO 00/06529 or WO 02/06246 A1 (both Merck); an NS5B inhibitor as disclosed in WO 03/000254 (Japan Tobacco); an NS5B inhibitor as disclosed in EP 1 256,628 A2 (Agouron); JTK-002 (Japan Tobacco); JTK-109 (Japan Tobacco); and the like.

### Ribavirin

In some embodiments, the at least one additional suitable therapeutic agent includes ribavirin. Thus, in some embodiments, a subject method comprises administering effective amounts of: i) an agent (e.g., an Hsp90 inhibitor) that reduces the activity of a host cell protein required for maturation of a viral protein; and ii) ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). The ribavirin may be administered orally in capsule or tablet form, or in the same or different administration form and in the same or different route as the agent that reduces the activity of a host cell protein that is required for maturation of one or more proteins encoded by an RNA virus. Of course, other types of administration of both medicaments, as they become available are contemplated, such as by nasal spray, transdermally, by suppository, by sustained release dosage form, etc. Any form of administration will work so long as the proper dosages are delivered without destroying the active ingredient.

Ribavirin is generally administered in an amount ranging from about 400 mg to about 1200 mg, from about 600 mg to about 1000 mg, or from about 700 to about 900 mg per day. In some embodiments, ribavirin is administered throughout the entire course of active agent (e.g., Hsp90 inhibitor) therapy. In other embodiments, ribavirin is administered only during the first period of time. In still other embodiments, ribavirin is administered only during the second period of time.

In some embodiments, the at least one additional suitable therapeutic agent includes levovirin. Thus, in some embodiments, a subject method comprises administering effective amounts of: i) an agent (e.g., an Hsp90 inhibitor) that reduces the activity of a host cell protein required for maturation of a viral protein; and ii) levovirin. Levovirin is the L-enantiomer of ribavirin and has the following structure:

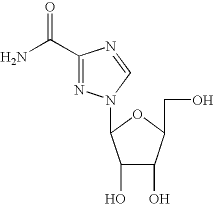

In some embodiments, the at least one additional suitable therapeutic agent includes viramidine. Thus, in some embodiments, a subject method comprises administering effective amounts of: i) an agent (e.g., an Hsp90 inhibitor) that reduces the activity of a host cell protein required for maturation of a viral protein; and ii) viramidine. Viramidine is a 3-carboxamidine derivative of ribavirin, and acts as a prodrug of ribavirin. Viramidine has the following structure:

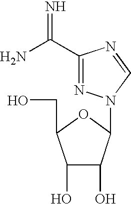

### Peptidyl-Prolyl Isomerase Inhibitors

In some embodiments, an agent that reduces the activity of a host cell protein that is required for maturation of one or more proteins encoded by the RNA virus is administered in conjunction with administration of a peptidyl-prolyl isomerase (PPI) inhibitor. PPIs include cyclophilins; and FK506 binding protein. Thus, in some embodiments, a subject method comprises administering effective amounts of: i) an agent (e.g., an Hsp90 inhibitor) that reduces the activity of a host cell protein required for maturation of a viral protein; and ii) a PPI inhibitor, e.g., an inhibitor of a cyclophilin or an FK506 binding protein. Suitable PPI inhibitors include, but are not limited to, cyclosporin (also known as Ciclosporin); FK506; ascomycin; rapamycin (see, e.g., U.S. Pat. No. 3,929,992; and U.S. Pat. No. 3,993,749); a rapamycin derivative or analog (see, e.g., U.S. Pat. No. 7,300,942; and U.S. Pat. No. 5,665,772); a cyclosporin-FK506 hybrid macrocyclic compound; FK520; FK523; FK525; antascomicin; meridamycin; tsukubamycin; 40-O-(2-hydroxy)ethyl rapamycin; 33-epi-chloro-33-desoxy-ascomycin; Cyclosporin A; Cyclosporin G, [0-(2-hydroxyethyl)-(D)Ser]⁸-Ciclosporin; and [3′-deshydroxy-3′-keto-MeBmt]¹-[Val]²-Ciclosporin; and the like.

### Formulations, Dosages, Routes of Administration

An active agent (also referred to herein as “drug”) is formulated with one or more pharmaceutically acceptable excipients. As noted above, “active agents” include, e.g., an Hsp90 inhibitor, and in some embodiments, further include a second active agent such as an HDAC inhibitor, an NS3 inhibitor, etc. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, an active agent may be administered to the host using any convenient means capable of resulting in the desired reduction in viral titers, symptoms of viral infection, etc. Thus, the active agent can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, an active agent may be administered in the form of their pharmaceutically acceptable salts, or an active agent may be used alone or in appropriate association, as well as in combination, with other pharmaceutically active compounds. The following methods and excipients are merely exemplary and are in no way limiting.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise an active agent in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for an active agent depend on the particular compound employed and the effect to be achieved, and the pharmacodynamics associated with each compound in the host.

An active agent can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles. An active agent is in some embodiments formulated into a preparation suitable for injection (e.g., subcutaneous, intravenous, intramuscular, intradermal, transdermal, or other injection routes) by dissolving, suspending or emulsifying the agent in an aqueous solvent (e.g., saline, and the like) or a nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, an active agent can be formulated alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives, and flavoring agents. For enteral delivery, a subject formulation will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit, and shellac.

As one non-limiting example of a suitable oral formulation, an active agent can be formulated together with one or more pharmaceutical excipients and coated with an enteric coating, as described in U.S. Pat. No. 6,346,269. For example, a solution comprising a solvent, an active agent, and a stabilizer is coated onto a core comprising pharmaceutically acceptable excipients, to form an active agent-coated core; a sub-coating layer is applied to the active agent-coated core, which is then coated with an enteric coating layer. The core generally includes pharmaceutically inactive components such as lactose, a starch, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin, and castor oil. Suitable solvents for the active agent include aqueous solvents. Suitable stabilizers include alkali-metals and alkaline earth metals, bases of phosphates and organic acid salts and organic amines. The sub-coating layer comprises one or more of an adhesive, a plasticizer, and an anti-tackiness agent. Suitable anti-tackiness agents include talc, stearic acid, stearate, sodium stearyl fumarate, glyceryl behenate, kaolin and aerosil. Suitable adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit™, copolymer of methyl acrylic acid/methyl methacrylate with polyvinyl acetate phthalate (PVAP). Suitable plasticizers include glycerin, polyethylene glycol, triethyl citrate, tributyl citrate, propanyl triacetate and castor oil. Suitable enteric-soluble coating material include hydroxypropyl methylcellulose acetate succinate (HPMCAS), hydroxypropyl methyl cellulose phthalate(HPMCP), cellulose acetate phthalate (CAP), polyvinyl phthalic acetate (PVPA), Eudragit™ and shellac.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Dosages

In some embodiments, an active agent is administered in an amount of from about 10 μg to about 500 mg per dose, e.g., from about 10 μg to about 20 μg, from about 20 μg to about 25 μg, from about 25 μg to about 50 μg, from about 50 μg to about 75 μg, from about 75 μg to about 100 μg, from about 100 μg to about 150 μg, from about 150 μg to about 200 μg, from about 200 μg to about 250 μg, from about 250 μg to about 300 μg, from about 300 μg to about 400 μg, from about 400 μg to about 500 μg, from about 500 μg to about 750 μg, from about 750 μg to about 1 mg, from about 1 mg to about 10 mg, from about 10 mg to about 25 mg, from about 25 mg to about 50 mg, from about 50 mg to about 100 mg, from about 100 mg to about 200 mg, from about 200 mg to about 300 mg, from about 300 mg to about 400 mg, or from about 400 mg to about 500 mg per dose.

In some embodiments, an active agent is administered in a dose that is lower than the dose of the agent that would be used to treat a cancer, and above a threshold level that is effective in treating an RNA viral infection. For example, in some embodiments, an active agent is administered in an amount of from about 10 mg/m² per dose to about 150 mg/m² per dose, e.g., from about 10 mg/m² per dose to about 15 mg/m² per dose, from about 15 mg/m² per dose to about 20 mg/m² per dose, from about 20 mg/m² per dose to about 25 mg/m² per dose, from about 25 mg/m² per dose to about 30 mg/m² per dose, from about 30 mg/m² per dose to about 35 mg/m² per dose, from about 35 mg/m² per dose to about 40 mg/m² per dose, from about 40 mg/m² per dose to about 50 mg/m² per dose, from about 50 mg/m² per dose to about 60 mg/m² per dose, from about 60 mg/m² per dose to about 70 mg/m² per dose, from about 70 mg/m² per dose to about 80 mg/m² per dose, from about 80 mg/m² per dose to about 90 mg/m² per dose, from about 90 mg/m² per dose to about 100 mg/m² per dose, from about 100 mg/m² per dose to about 110 mg/m² per dose, from about 110 mg/m² per dose to about 120 mg/m² per dose, from about 120 mg/m² per dose to about 130 mg/m² per dose, from about 130 mg/m² per dose to about 140 mg/m² per dose, or from about 140 mg/m² per dose to about 150 mg/m² per dose.

In some embodiments, an active agent is administered in an amount of from about 10 mg/m² per week to about 200 mg/m² per week, e.g., from about 10 mg/m² per week to about 15 mg/m² per week, from about 15 mg/m² per week to about 20 mg/m² per week, from about 20 mg/m² per week to about 25 mg/m² per week, from about 25 mg/m² per week to about 30 mg/m² per week, from about 30 mg/m² per week to about 35 mg/m² per week, from about 35 mg/m² per week to about 40 mg/m² per week, from about 40 mg/m² per week to about 50 mg/m² per week, from about 50 mg/m² per week to about 60 mg/m² per week, from about 60 mg/m² per week to about 70 mg/m² per week, from about 70 mg/m² per week to about 80 mg/m² per week, from about 80 mg/m² per week to about 90 mg/m² per week, from about 90 mg/m² per week to about 100 mg/m² per week, from about 100 mg/m² per week to about 110 mg/m² per week, from about 110 mg/m² per week to about 120 mg/m² per week, from about 120 mg/m² per week to about 130 mg/m² per week, from about 130 mg/m² per week to about 140 mg/m² per dose, from about 140 mg/m² per week to about 150 mg/m² per week, from about 150 mg/m² per week to about 160 mg/m² per week, from about 160 mg/m² per week to about 170 mg/m² per week, from about 170 mg/m² per week to about 180 mg/m² per week, from about 180 mg/m² per week to about 190 mg/m² per week, or from about 190 mg/m² per week to about 200 mg/m² per week.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, multiple doses of an active agent are administered. The frequency of administration of an active agent can vary depending on any of a variety of factors, e.g., severity of the symptoms, etc. For example, in some embodiments, an active agent is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). In some embodiments, active agent is administered continuously.

The duration of administration of an active agent, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, an active agent is administered for the lifetime of the individual.

In some embodiments, administration of an active agent is discontinuous, e.g., an active agent is administered for a first period of time and at a first dosing frequency; administration of the active agent is suspended for a period of time; then the active agent is administered for a second period of time for a second dosing frequency. The period of time during which administration of the active agent is suspended can vary depending on various factors, e.g., patient response; and will generally range from about 1 week to about 6 months, e.g., from about 1 week to about 2 weeks, from about 2 weeks to about 4 weeks, from about one month to about 2 months, from about 2 months to about 4 months, or from about 4 months to about 6 months, or longer. The first period of time may be the same or different than the second period of time; and the first dosing frequency may be the same or different than the second dosing frequency.

### Routes of Administration

An active agent is administered to an individual using any available method and route suitable for drug delivery, including systemic and localized routes of administration.

Conventional and pharmaceutically acceptable routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. The compound can be administered in a single dose or in multiple doses.

An active agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations. Inhalational routes of delivery are also contemplated, e.g., where the virus is one that infects the airways, lungs, etc.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

### Subjects Suitable for Treatment

A subject treatment method generally involves administering to an individual in need thereof an effective amount of an active agent that reduces the activity of a host cell protein that is required for maturation of a viral protein, e.g., an agent that inhibits a heat shock protein or chaperone that facilitates maturation of one or more viral proteins. Individuals in need of treatment with a subject treatment method include: a) individuals who have been exposed to a virus, but who have not yet been infected; b) individuals who have been infected with a virus, and who have not been treated with any anti-viral agent (e.g., infected and treatment naïve individuals); c) individuals who have been infected with a virus, who have been treated with an anti-viral agent other than an Hsp90 inhibitor, and who have developed resistance to the anti-viral agent other than an Hsp90 inhibitor.

In some embodiments, individuals in need of treatment with a subject include: a) individuals who have been exposed to an RNA virus, but who have not yet been infected with the RNA virus; b) individuals who have been infected with an RNA virus, and who have not been treated with any anti-viral agent for the RNA virus infection (e.g., infected and treatment naïve individuals); c) individuals who have been infected with a RNA virus, who have been treated with an anti-viral agent other than an agent that reduces the activity of a host cell protein required for maturation of an RNA viral protein, and who have developed resistance to the anti-viral agent.

### Picornavirus

In some embodiments, individuals in need of treatment with a subject include: a) individuals who have been exposed to a picornavirus, but who have not yet been infected with the picornavirus; b) individuals who have been infected with a picornavirus, and who have not been treated with any anti-viral agent for the picornavirus infection (e.g., infected and treatment naïve individuals); c) individuals who have been infected with a picornavirus, who have been treated with an anti-viral agent other than an Hsp90 inhibitor, and who have developed resistance to the anti-viral agent other than an Hsp90 inhibitor.

### Flavivirus

In some embodiments, individuals in need of treatment with a subject include: a) individuals who have been exposed to West Nile Virus (WNV), but who have not yet been infected with the WNV; b) individuals who have been infected with WNV, and who have not been treated with any anti-viral agent for the WNV infection (e.g., infected and treatment naïve individuals); c) individuals who have been infected with WNV, who have been treated with an anti-viral agent other than an Hsp90 inhibitor, and who have developed resistance to the anti-viral agent other than an Hsp90 inhibitor.

In some embodiments, individuals in need of treatment with a subject include: a) individuals who have been exposed to Yellow Fever Virus (YFV), but who have not yet been infected with the YFV; b) individuals who have been infected with YFV, and who have not been treated with any anti-viral agent for the YFV infection (e.g., infected and treatment naïve individuals); c) individuals who have been infected with YFV, who have been treated with an anti-viral agent other than an Hsp90 inhibitor, and who have developed resistance to the anti-viral agent other than an Hsp90 inhibitor.

In some embodiments, individuals in need of treatment with a subject include: a) individuals who have been exposed to Dengue virus, but who have not yet been infected with the Dengue virus; b) individuals who have been infected with Dengue virus, and who have not been treated with any anti-viral agent for the Dengue virus infection (e.g., infected and treatment naïve individuals); c) individuals who have been infected with Dengue virus, who have been treated with an anti-viral agent other than an Hsp90 inhibitor, and who have developed resistance to the anti-viral agent other than an Hsp90 inhibitor.

In some embodiments, individuals in need of treatment with a subject include: a) individuals who have been exposed to Hepatitis C Virus (HCV), but who have not yet been infected with the HCV; b) individuals who have been infected with HCV, and who have not been treated with any anti-viral agent for the HCV infection (e.g., infected and treatment naïve individuals); c) individuals who have been infected with HCV, who have been treated with an anti-viral agent other than an Hsp90 inhibitor, and who have developed resistance to the anti-viral agent other than an Hsp90 inhibitor. Where the individual is infected with HCV, the HCV can be any of a number of genotypes, subtypes, or quasispecies, including, e.g., genotype 1, including 1a and 1b, 2, 3, 4, 6, etc. and subtypes (e.g., 2a, 2b, 3a, 4a, 4c, etc.), and quasispecies.

In some embodiments, the individual is a treatment failure patient, e.g., an individual who is infected with HCV and who failed treatment for the HCV infection, where the treatment regimen involved treatment with an agent other than an agent that reduces the activity of a host cell protein that is required for maturation of a viral protein. The term “treatment failure patients” (or “treatment failures”) as used herein generally refers to HCV-infected patients who failed to respond to previous therapy for HCV (referred to as “non-responders”) or who initially responded to previous therapy, but in whom the therapeutic response was not maintained (referred to as “relapsers”). Relapsers include individuals infected with an HCV that has become resistant to a previous treatment regimen, e.g., where the treatment regimen involved treatment with an agent other than an agent that reduces the activity of a host cell protein that is required for maturation of a viral protein. Previous treatment regimens can include, e.g., IFN-α treatment, ribavirin treatment, or an IFN-α/ribavirin combination treatment.

As non-limiting examples, individuals suitable for treatment with a subject method can have, before treatment with a subject method, an HCV titer of at least about 10⁵, at least about 5×10⁵, or at least about 10⁶, genome copies of HCV per milliliter of serum.

### Influenza Virus

In some embodiments, individuals in need of treatment with a subject include: a) individuals who have been exposed to an influenza virus, but who have not yet been infected with the influenza virus; b) individuals who have been infected with an influenza virus, and who have not been treated with any anti-viral agent for the influenza virus infection (e.g., infected and treatment naïve individuals); c) individuals who have been infected with an influenza virus, who have been treated with an anti-viral agent other than an Hsp90 inhibitor, and who have developed resistance to the anti-viral agent other than an Hsp90 inhibitor.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Inhibition of Picornavirus

Hsp90 inhibitors impaired the replication of three major picornavirus pathogens in tissue culture: poliovirus, the agent of poliomyelitis; rhinovirus, the agent of the common cold; and coxsackievirus. Strikingly, poliovirus was unable to develop escape mutants resistant to an Hsp90 inhibitor, even though its rapid replication rate and high mutation frequency (10⁶ times higher than that of DNA based genomes) enabled the isolation of drug resistant poliovirus variants to virtually all other antiviral compounds tested to date. These results suggest that stringent constraints prevent proteins from being able to evolve folding pathways that bypass their Hsp90 requirement. Importantly, this finding uncovered a target for antiviral therapies which may be refractory to development of drug resistance in vivo. Indeed, it was found that administration of Hsp90 inhibitors to infected animals drastically reduced poliovirus replication without eliciting viral drug resistance.

### Materials and Methods

### Cells, Viruses, and Reagents

HeLa S3 cells, TSA201, Vero and human foreskin fibroblasts were cultured under standard procedures. For experiments with human rhinovirus 14 (HRV14) cells were grown at 33° C. Geldanamycin (GA), 17-(Allylamino)-17-demethoxygeldanamycin (17AAG, LC laboratories), Lactacystin (LC, EMD biosciences), N-Acetyl-L-leucyl-L-leucyl-L-norleucinal (ALLN, Calbiochem), and Brefeldin A (BFA, LC laboratories) were dissolved in DMSO and E64 (Boehringer Mannheim) in 70% ethanol. GA was obtained from the National Cancer Institute, Drug Synthesis and Chemistry Branch, Developmental Therapeutics Program, Division of Cancer Treatment and Diagnosis. All GA experiments were done under dim light conditions. Poliovirus Mahoney type 1 strain (PV) was generated from plasmid pRib (+)XpA as previously described (Herold and Andino (2000) J. Virol. 74:6394). HRV14 was obtained from American Tissue Culture Collection. The coxsackievirus B3 (CVB3) construct (Klump et al. (1990) J. Virol. 64:1573), was generated as described (Herold and Andino (2000) supra). Vaccinia virus P1 (VV-P1) has been described (Ansardi et al. (1991) J. Virol. 65:2088). Egg phosphatidylcholine was purchased from Avanti Polar Lipids.

### Viral Infections

PV, CVB3 or HRV14 were allowed to adsorb to cells for 30 minutes, at 37° C. (for PV, CVB3) or 33° C. (for HRV14), after which cells were washed with PBS and incubated in culture media. VV-P1 infections were carried out for 1.5 hour at 37° C. Both VV-P1 and CVB3 infections were carried out in media containing low serum concentrations (2% FCS).

### Effect of GA on Viral Replication in Cultured Cells

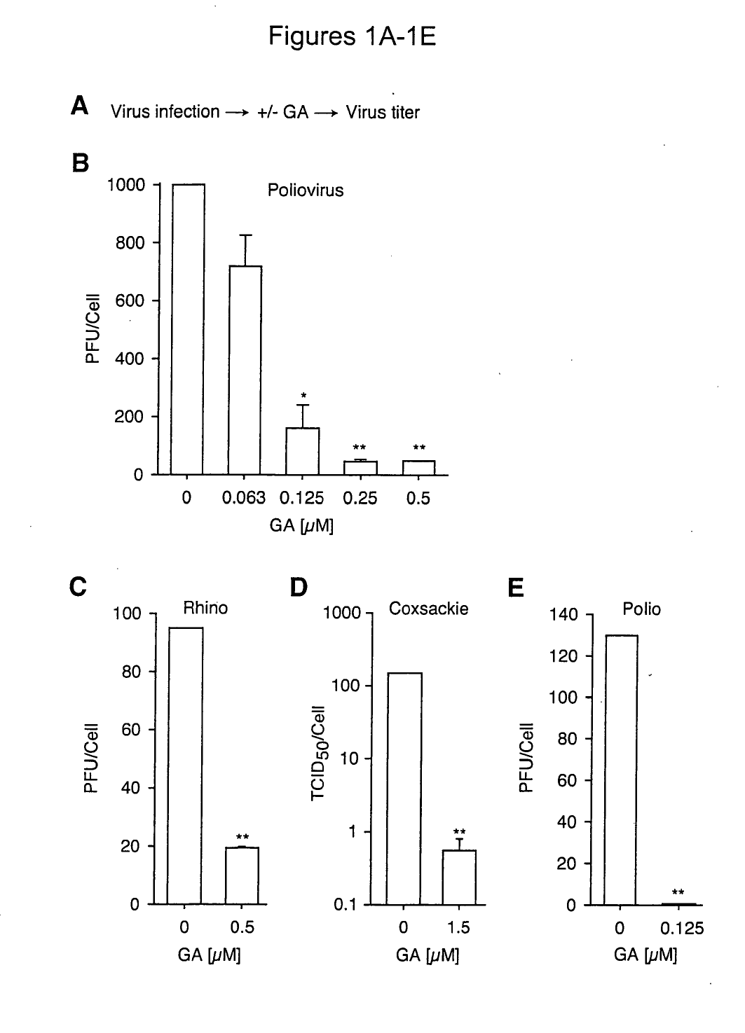

Hela S3, TSA201 or HFF cells were infected at a multiplicity of infection (MOI) of 1-5 and plated in the presence or absence of GA. A 45 minute pre-incubation step with GA was included in FIGS. 1B and 1C. Virus production was measured by standard plaque assay (PV, HRV14) or end-point titration on Vero cells (CVB3).

### In Vitro Transcription and RNA Electroporation

Poliovirus genomic or replicon RNA were transcribed from pRib (+)XpA or pRib (+)RLuc plasmids, respectively, as previously described (Herold and Andino (2000) supra). For in vitro translation, P1 was amplified from pRib (+)XpA by PCR and cloned into pCDNA3.1 (+) using HindIII and XhoI restriction sites. Capped RNA was generated using the MegaScript T7 kit (Ambion) after linearization with XhoI following manufacturer's protocol. For electroporations, HeLa S3 cells (4×10⁶) were pulsed with 10 μg of RNA in 0.8 ml Ca²⁺/Mg²⁺ free PBS using a BTX electroporator set to 950 μf, 128 Ω, and 300 V in a 0.4 cm cuvette. When indicated, GA was used at 2 μM concentrations.

### Radiolabeling and Immunoprecipitation

Cells were infected at an MOI>25. GA (0.5 μM) was added 2 hours post infection and maintained for the remainder of the experiment. For steady state pulse experiments, cells were incubated with 30 μCi/mL ³⁵S methionine/cysteine for 2 hours in media lacking these amino acids. Cells were then washed in PBS, lysed in lysis buffer (25 mM TRIS pH 7.5, 150 mM NaCl, 1% NP40, protease inhibitor cocktail (Sigma)), and analyzed by 12% sodium dodecyl sulfate polyacrylamide gel electrophoresis (SDS-PAGE) and autoradiography on a Typhoon PhosphoImager (Amersham Biosciences). For pulse chase experiments, cells were starved for 15 minutes at 3 hours post-infection, incubated in media containing ³⁵S methionine/cysteine for 5 minutes, and chased in media containing the amino acids for the indicated time prior to analysis as above. For experiments with VV-P1, cells were infected at an MOI of 10, radiolabeled as above for 1.5 hours, washed, and media containing LC (2004), ALLN (10 μM), or E64 (25 μM) was added. GA (0.5 μM) was added 3 hours later and cells incubated for an additional 3 hours prior to lysis in RIPA buffer and immunoprecipitation with polyclonal αVirion-N1 antibodies. All quantifications were performed using ImageQuant software (Amersham Biosciences).

### Chaperone Immunoprecipitations

Confluent 10 cm dishes were infected with poliovirus at an multiplicity of infection (MOI) of 50. Dimethyl sulfoxide (DMSO) or GA (1 μM) were added 2 hours post infection and maintained for the remainder of the experiment. Four hours post infection, cells were starved and radiolabeled for 30 minutes as above prior to lysis in Hsp90 Lysis Buffer (20 mM Hepes pH 7.5, 100 mM NaCl, 20 mM Sodium Molybdate, 5 mM EDTA, 10% glycerol, 0.01% NaN₃, protease inhibitor cocktail (Sigma)) containing 10 mg/mL BSA. Nuclei were removed by centrifugation and supernatants incubated with antibodies to Hsp90 (SPA840, Stressgen), p23 (JJ3), or a control antibody, for 1 hour on ice. Lysates were then incubated with protein G-sepharose (Amersham Biosciences) for 45 minutes, washed 4 times in Hsp90 buffer, and analyzed by 12% SDS-PAGE and autoradiography.

### In Vitro Translation and 3C^{pro}-HA Purification

In vitro transcribed P1 RNA was translated in Flexi Rabbit Reticulocyte Lysate (Promega) following manufacturer instructions. Reactions were stopped by incubation with cycloheximide (0.1 mg/ml) and RNase A (80 μg/ml) for 5 minutes followed by addition of DMSO, GA (0.5 mM), or EDTA (15 mM) for 10 minutes at 30° C. Bacterially purified 3C^{pro} was then added (0.6 mg/mL) for the indicated time and processing analyzed by SDS-PAGE and autoradiography as above. 3C^{pro} was purified via a C terminal HIS₆ tag as described below.

### Protein Purification

To purify the protease, 3C^{pro} was PCR amplified from pRib (+)XpA with NcoI and XhoI restriction sites, cloned into a pET28-a (+) vector (Novagen) in frame with a C terminal HIS₆ tag, and used to transform BL21 (DE3) bacteria. At OD600, a 1.2 L culture was induced with 100 μM IPTG for 3 hours at 37° C. Purification was performed using TALON metal affinity resin (BD Biosciences) following standard protocols. Purified protein was dialyzed against dialysis buffer (20 mM Hepes, 100 mM NaCl, 10% glycerol, 1 mM DTT, 1 mM EDTA, (pH 7.6)) and frozen at −80° C.

### Animal Experiments

On day 0, 6-10 week old male and female cPVR transgenic mice (Crotty et al. (2002) J. Gen. Virol. 83:1707) were injected with the Hsp90 inhibitors or vehicle (i.p.) and infected 4-6 hours later with 10⁷ plaque-forming units (PFU) of poliovirus by tail vein injection. Hsp90 inhibitors or vehicle alone were administered daily for the subsequent 3 days. Virus production was determined on day 5 as previously described (Crotty et al. (2002) supra). For GA experiments, each injection contained 2.5 μg (0.1 mg/kg) of GA in 5 μL DMSO and 45 μl of PEG400:H₂O (1:1). For experiments with 17AAG, each injection contained either 0.05 mg (2.5 mg/kg) or 0.5 mg (25 mg/kg) of 17AAG in 5 μL of DMSO and 45 μL of 2% egg phosphatidylcholine and 5% dextrose (NSC 704057). All animal experiments were in accordance with institutional guidelines.

### Statistical Analysis

All data for in vitro and tissue culture experiments are represented as the mean of the indicated number of experiments. Error bars indicate SEM. Significance was tested with a two-tail t-test. For in vivo experiments, a two-tail Wilcoxon two sample test was employed using the NPAR1WAY procedure on SAS software.

### Results

### Pharmacological Inhibition of Hsp90 Impairs Viral Replication in Cultured Cells

The effect of pharmacologically inhibiting Hsp90 on the replication of three pathogens of the picornavirus family—poliovirus, rhinovirus and coxsackievirus (FIG. 1A—was tested. Geldanamycin (GA), a specific Hsp90 inhibitor, was used to inhibit Hsp90. HeLa S3 cells were infected with poliovirus in the presence or absence of increasing concentrations of GA and virus production measured at 7 hours post-infection. GA treatment inhibited poliovirus replication in a dose dependent manner, with maximal inhibition of 95% and an IC₅₀ of 0.11 μM (+/−0.026) relative to DMSO treated controls (FIG. 1B). A similar antiviral effect by GA was observed in TSA201 cells. GA treatment also inhibited the replication of rhinovirus (FIG. 1C) and coxsackievirus (FIG. 1D). It has been reported that the Hsp90 machinery of transformed cells is more susceptible to GA than that of untransformed cells. Kamal et al. (2003) Nature 425:407. To evaluate if GA also possess antiviral activity in untransformed cells, it was examined whether GA can inhibit poliovirus replication in primary human foreskin fibroblasts (FIG. 1E). Strikingly, the inhibitory effect of GA on poliovirus replication was even stronger in these cells than in HeLa S3 or TSA201 cells (FIG. 1E, >99% inhibition at 0.1 μM). These results indicate that Hsp90 function is required for picornavirus replication.

FIG. 1. The Hsp90 inhibitor GA reduces picornavirus replication in cultured cells. (A) Outline of the experiment. (B-E) Effect of GA on poliovirus (B, E), rhinovirus (C) and coxsackievirus (D) production in HeLa S3 cells (B-D) or primary human foreskin fibroblasts (E). Data are represented as number of Plaque Forming Units (PFU) or 50% Tissue Culture Infective Dose (TCID₅₀) produced per cell and, for comparison reasons, standardized between experiments so as to yield the same number of PFU or TCID₅₀/Cell for DMSO treated conditions. Results indicate mean and SEM of three independent experiments. * p<0.05, ** p<0.001 relative to DMSO treated condition by t-test.

### Geldanamycin Decreases Production of Mature Capsid Proteins

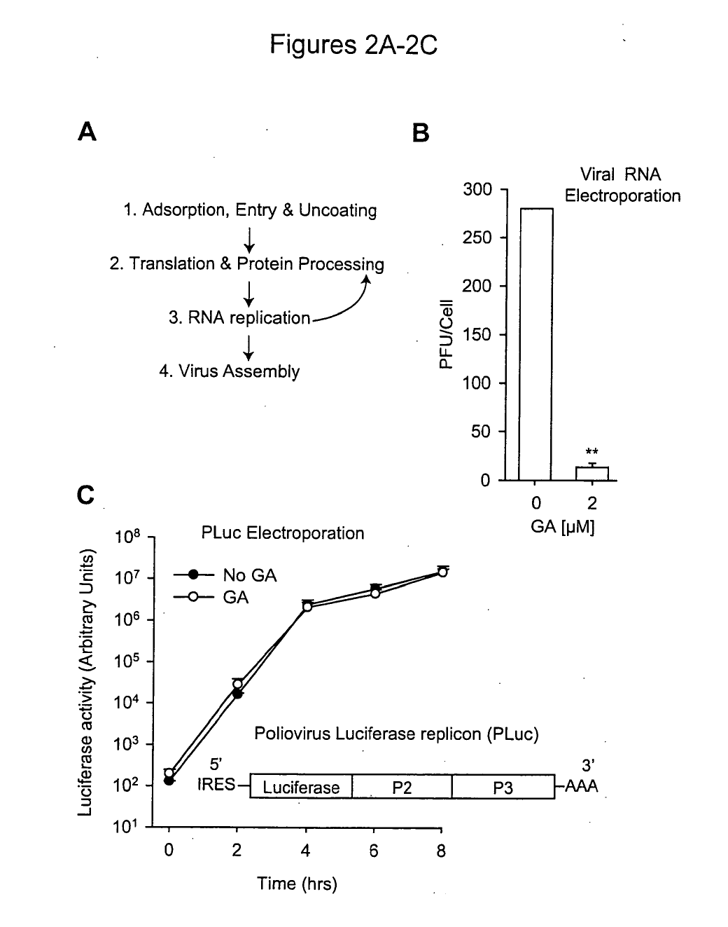

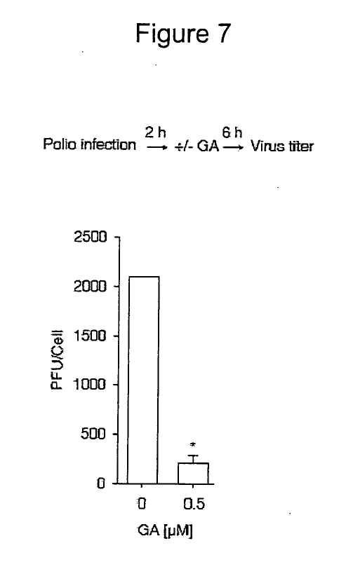

The molecular basis for the anti-picornavirus activity of GA was defined by systematically examining its effect on distinct steps in the viral life cycle (FIG. 2A). It was first tested whether GA affects the early steps of viral replication (FIG. 2B). To bypass the viral entry and uncoating steps, the viral genomic RNA (vRNA) was directly introduced into cells; this allowed one to measure the effect of GA on subsequent steps in virus production (FIG. 2B). GA treatment inhibited virus production in vRNA transfected cells to the same degree as when cells were infected with intact virus (95% inhibition, FIG. 2B and FIG. 1B), indicating that GA acts downstream of these early steps. Consistent with this conclusion, GA treatment effectively inhibited virus production even when added two hours post-infection, at a stage subsequent to viral entry and uncoating (FIG. 7).

Following viral entry, the positive stranded genomic RNA is translated to produce the viral replication machinery, which in turn synthesizes more genomic RNA (FIG. 2A). To examine whether GA treatment targets translation and/or replication of the viral genome, a poliovirus replicon, PLuc, which carries the firefly luciferase gene in lieu of the capsid coding sequence P1 (Herold and Andino (2000) supra) was employed. Since PLuc translates and replicates like wildtype virus, luciferase activity provides a quantitative measure of viral translation and RNA replication (Andino et al. (1993) EMBO J. 12:3587). Importantly, GA treatment did not affect luciferase production in PLuc transfected cells (FIG. 2C). Thus, GA does not inhibit translation or replication of the viral genome.

The poliovirus genome encodes a single open reading frame that is translated to yield a single poly-protein. Viral-encoded proteases, such as 2A^{pro}, 3C^{pro} or its precursor 3CD, liberate three proteins P1, P2, and P3, which are further processed to generate the mature viral proteins. Because the PLuc replicon encodes all viral proteins except for the capsid precursor P1, these results suggest that Hsp90 is not required for the function of P2 and P3 derived proteins but rather participates in P1 function. P1 maturation involves processing into three capsid proteins: VP0, VP3, and VP1 (see FIG. 2D). VP0 is itself a precursor to VP4 and VP2, but is only cleaved at a late stage of particle assembly, probably following genome encapsidation (Basavappa et al. (1994) Protein Sci. 3:1651).

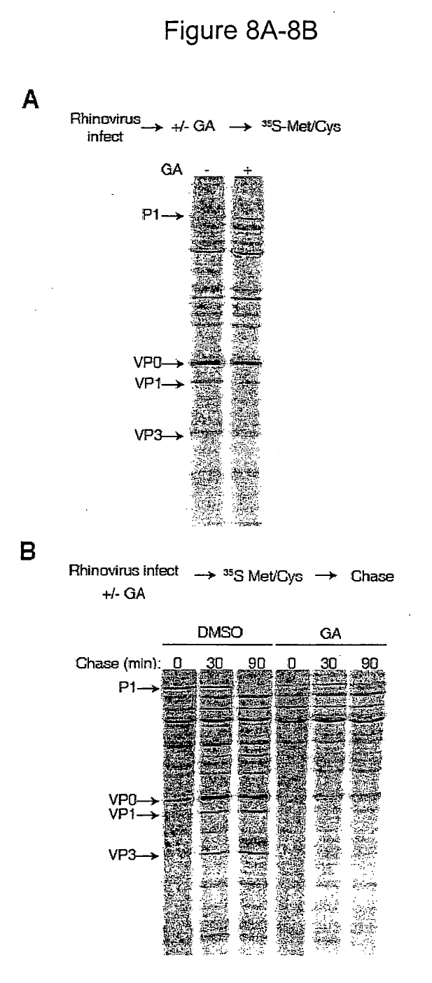

The effect of Hsp90 inhibition on the processing and maturation of viral proteins was further examined using ³⁵S-labeling of poliovirus-infected cells (FIGS. 2E and 2F). Because poliovirus efficiently shuts-off cellular translation, only viral proteins are radiolabeled under these conditions. As expected, both precursors and mature viral proteins were produced in control cells; on the other hand, treatment with GA produced a significant reduction in P1-derived capsid proteins (FIGS. 2E and 2F). However, GA treatment did not affect processing of P2 or P3, in agreement with our findings using the PLuc replicon (FIG. 2C). Of note, GA also impaired P1 processing in rhinovirus-infected cells (FIGS. 8A and 8B), suggesting a conserved mode of action for GA within the picornavirus family.

FIG. 2. Inhibition of Hsp90 specifically affects production of mature capsid proteins. (A) Schematic representation of picornavirus life-cycle. (B) GA inhibits poliovirus replication from a transfected infectious genomic RNA. Data represents the mean and SEM of three independent experiments. (C) GA does not inhibit translation and replication of a poliovirus luciferase replicon (PLuc), in which the capsid coding sequence is replaced with luciferase (Herold and Andino (2000) supra). The time course of luciferase activity reports on viral translation and replication. Data shows mean and SEM of three independent experiments. (D) Poliovirus encoded polyprotein, highlighting the processing events for the capsid precursors. (E, F) GA decreases capsid protein production (E) Steady-state ³⁵S-labeling of poliovirus proteins from infected cells grown in the presence or absence of GA. Total cytoplasmic extracts (lanes 3 & 4) and immunoprecipitated capsid proteins (lanes 1 & 2) separated by SDS-PAGE were visualized by autoradiography. P1-derived (labeled arrows) and P2- and P3-derived proteins (arrowheads) are indicated. (F) Relative band intensity of P1 and P1-derived capsid proteins in control and GA-treated cells. Data shows means and SEM of four independent experiments performed as in E. * p<0.05, ** p<0.001 relative to control treated cells by t-test.

FIG. 7. GA effectively inhibits virus production if added after viral entry and uncoating have occurred. HeLa S3 cells were infected with poliovirus at an MOI of 5. Two hours post-infection, cells were treated with GA or DMSO. Virus production was assayed after 6 hours by standard plaque assay. Data represents the mean number of PFU/cell and SEM from two independent experiments. * p<0.05 relative to DMSO treated condition by t-test.

FIGS. 8A and 8B. GA inhibits rhinovirus P1 processing. SDS-PAGE of rhinovirus proteins from steady state labeling (A) or pulse-chase analysis (B) of rhinovirus infected HeLa S3 cells grown in the presence of GA or DMSO.

### The Capsid Protein P1 is a Folding Substrate of Hsp90

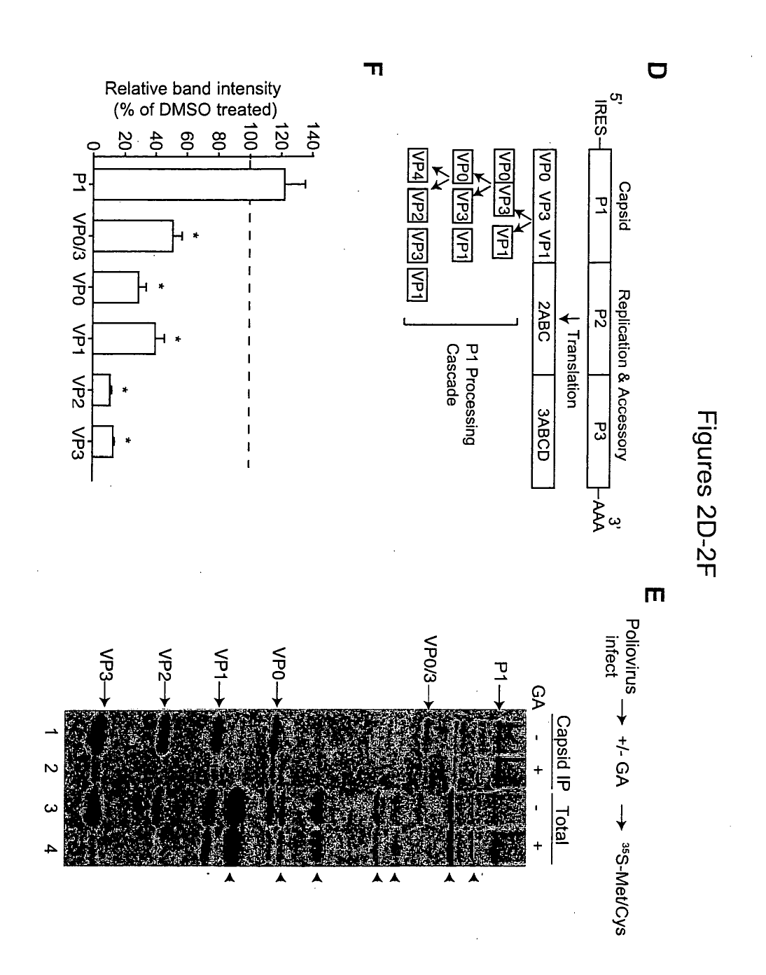

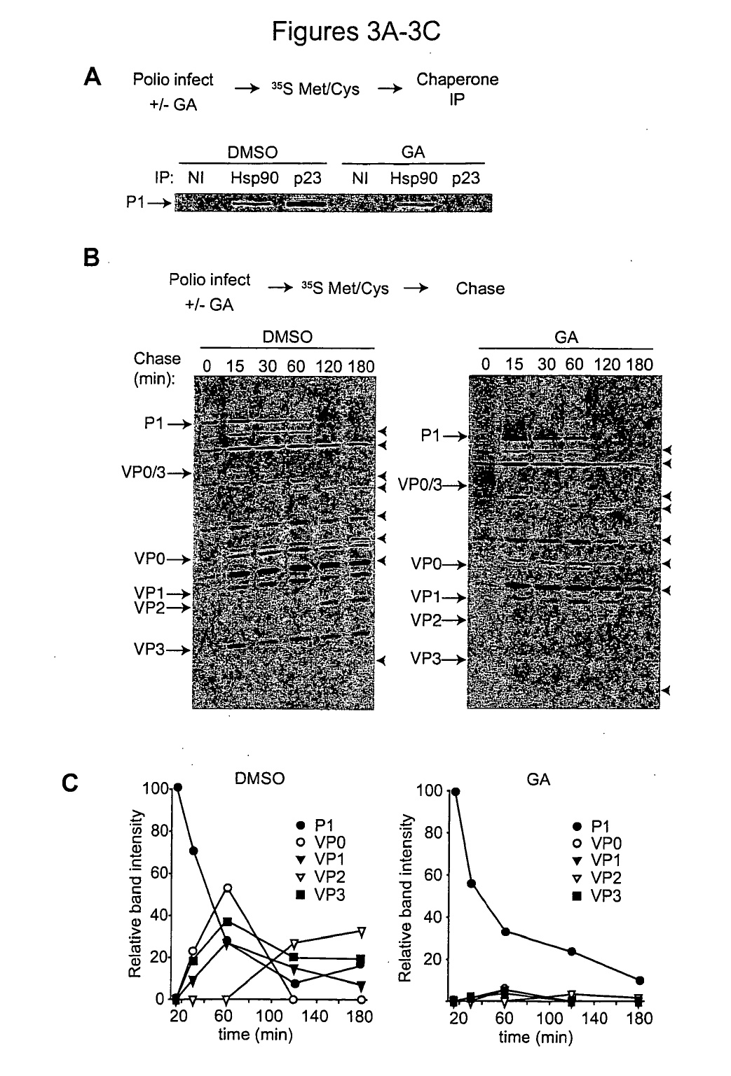

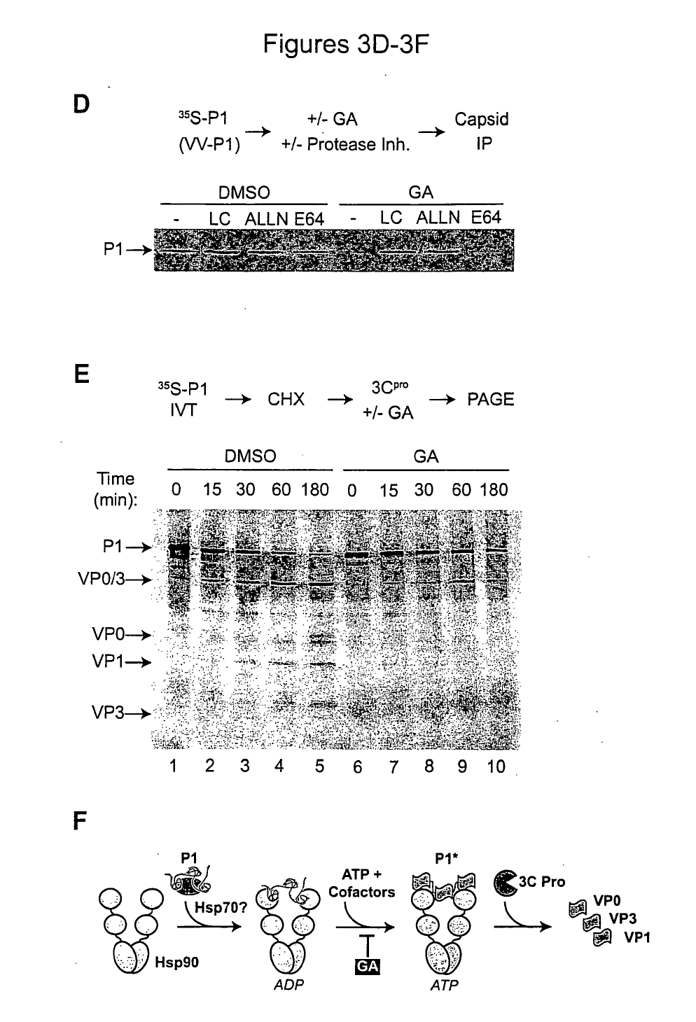

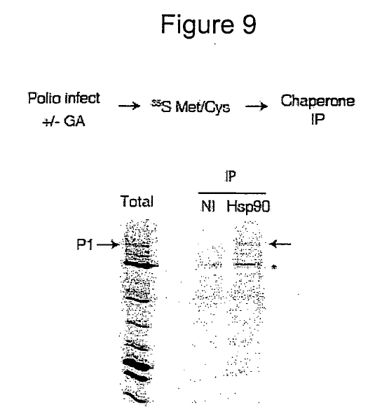

It was determined whether Hsp90 directly interacts with viral proteins. Co-immunoprecipitation experiments using ³⁵S-labeled poliovirus-infected cells indicated that Hsp90 and its co-chaperone p23 both associate with only one viral protein—the capsid precursor P1 (FIG. 3A and FIG. 9). This result is consistent with the specific effect of GA on capsid protein production in infected cells. Interestingly, treatment with GA did not disrupt the Hsp90-P1 interaction but abrogated the association of P1 with p23 (FIG. 3A). This result supports previous findings that GA inhibits the p23-Hsp90 interaction thus blocking progression through the Hsp90 chaperone cycle (Young et al. (2001) J. Cell Biol. 154:267; Picard (2002) Cell. Mol. Life Sci. 59:1640; Wegele et al. (2004) Rev. Physiol. Biochem. Pharmacol. 151:1; Ali et al. (2006) Nature 440:1013). It was concluded that p23 binds P1 through its nucleotide-dependent interaction with Hsp90. Furthermore, the action of p23 on Hsp90 is required for P1 maturation.

The effect of Hsp90 inhibition on P1 processing was next examined by pulse-chase analysis (FIGS. 3B and 3C). Poliovirus infected cells were subjected to a brief pulse of ³⁵S-methionine/cysteine to label viral protein precursors and then chased with unlabeled amino acids to examine their processing kinetics. GA treatment did not affect the appearance of some viral proteins, such as 3CD or 2C, consistent with a specific action on P1 (FIG. 3B, right arrow heads). Notably, the kinetics of P1 production also appeared unaffected by the presence of GA (FIGS. 3B and 3C). However, while P1 disappearance was largely unaffected by GA treatment, the appearance of the P1-derived mature capsid proteins was drastically reduced by Hsp90-inhibition (FIGS. 3B and 3C). Together, these results indicate that association with Hsp90 and its co-chaperone p23 are required for P1 processing into mature capsid proteins.

Why does P1 disappear following GA treatment without yielding capsid proteins? It was reasoned that if Hsp90 mediates P1 folding, its inhibition by GA could lead to P1 misfolding which in turn would target P1 for elimination by the cellular quality control machinery. To directly monitor the effect of Hsp90 inhibition on the fate of P1 in the absence of poliovirus-encoded processing proteases, P1 was expressed using a previously established vaccinia virus expression system (VV-P1) (Ansardi et al. (1991) J. Virol. 65:2088). In the absence of GA, P1 was stable; in contrast, it was readily degraded within 3 hours of GA treatment (FIG. 3D). Inhibition of the proteasome pathway with lactacystin (LC) or ALLN protected P1 from degradation (FIG. 3D). On the other hand, addition of the lysosomal protease inhibitor E64 resulted in minimal protection from degradation. Thus, disruption of the Hsp90-p23 complex results in P1 misfolding which targets it to the proteasome for degradation.

To better define the role of Hsp90 in P1 folding and maturation a cell free system was employed. ³⁵S-labeled P1 was generated by translation in rabbit reticulocyte lysate and processing of the capsid precursor into capsid proteins was then monitored following addition of purified 3C^{pro} (FIG. 3E, lanes 1-5). Inhibition of Hsp90 by GA significantly reduced P1 processing (FIG. 3E, lanes 6-10). Notably, in contrast to our observations in intact cells (FIGS. 3B and 3D), P1 did not disappear upon Hsp90 inhibition. This is consistent with findings that in translating reticulocyte lysates, proteasomal degradation is inhibited by free hemin (Haas and Rose (1981) Proc. Natl. Acad. Sci. USA 78:6845). Thus, even in the absence of proteasomal degradation, interaction with Hsp90 and p23 is still required for capsid protein maturation. These results suggest that Hsp90 does not simply protect P1 from proteasomal degradation but is required to fold it into a processing-competent conformation (FIG. 3F). Thus, inhibition of the Hsp90 chaperone cycle by GA leaves P1 in a misfolded conformation that cannot be recognized by 3C^{pro} and is instead degraded by the quality control systems.

FIG. 3. Hsp90 associates with the capsid precursor P1 and is required for its processing to mature capsid proteins. (A) Association of ³⁵S-labeled P1 with Hsp90 and its co-chaperone p23 in the presence or absence of GA, measured by immunoprecipitation; NI, non-immune control (B) Pulse-chase analysis of poliovirus proteins from infected cells grown in the presence or absence of GA. Total cytoplasmic extracts separated by SDS-PAGE were visualized by autoradiography. P1 derived (labeled arrows) and P2- and P3-derived proteins (arrowheads) are indicated. (C) Relative band intensity of P1 and P1-derived capsid proteins in control and GA-treated cells, calculated from B as percent of P1 at 15 minute chase time point. (D) GA treatment promotes P1 degradation by the proteasome. The effect of GA on degradation of ³⁵S-labeled P1, expressed in cells by infection with a recombinant vaccinia virus (VV-P1) (Ansardi et al. 1991) was examined in the presence or absence of the proteasome inhibitors LC and ALLN, and the lysosomal protease inhibitor E64. (E) Processing of in vitro translated P1 into capsid proteins by purified 3C^{pro} is blocked by GA even in the absence of proteasomal function. CHX, cycloheximide. (F) Role of Hsp90 in picornavirus capsid maturation. Hsp90 binds newly translated P1, probably in cooperation with Hsp70 (see Discussion and (Macejak and Sarnow 1992)). Together with ATP and its cofactors, such as p23, Hsp90 folds P1 to a processing-competent conformation (P1*) and protects it from proteasomal degradation. Upon cleavage by 3C^{Pro}, the mature capsid proteins no longer interact with Hsp90.

FIG. 9. Hsp90 binds only one viral protein in poliovirus infected cells. Immunoprecipitation of Hsp90 from poliovirus infected cells grown in the presence or absence of GA and radiolabeled with ³⁵S methionine/cysteine. NI, non-immune control. * indicates non-specific binding band. Arrow indicates migration of P1.

### The Virus Cannot Bypass the Hsp90 Requirement

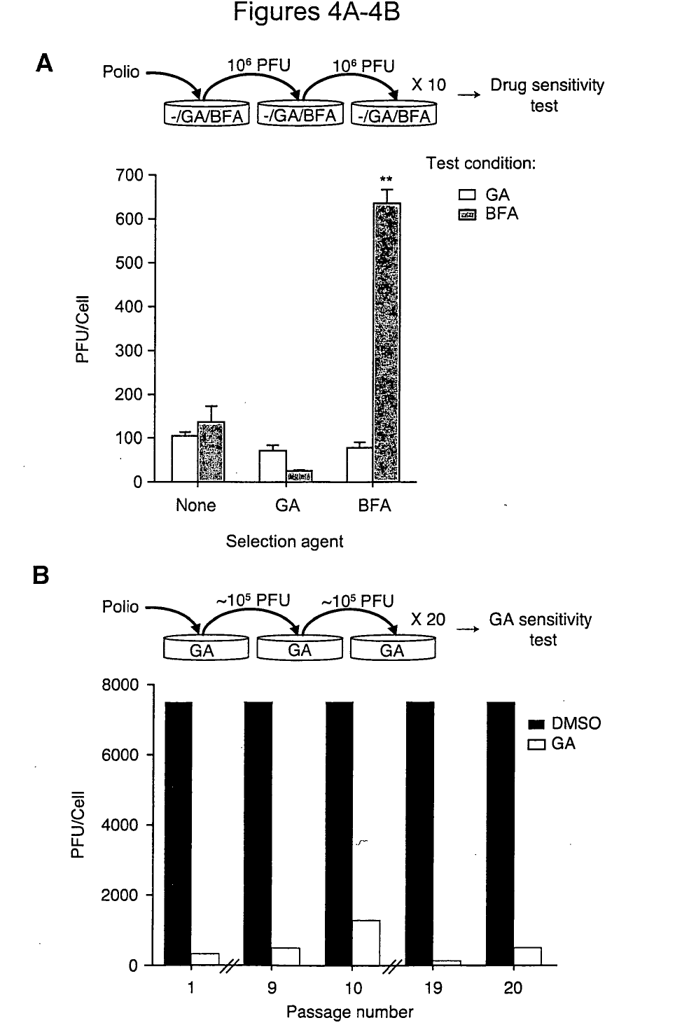

Having identified Hsp90 as essential for folding of a single protein in the picornavirus proteome, it was examined whether the evolutionary capacity of poliovirus can be exploited to drive P1 to fold via an Hsp90-independent pathway. To force the emergence of Hsp90-independent viral variants, poliovirus was subjected to serial passage in the presence of GA (FIG. 4A). This approach was found to yield resistance to a diverse array of antiviral compounds in fewer than six passages (Gitlin et al. (2002) Nature 418:430; Crotty et al. (2004) J. Virol. 78:3378; Vignuzzi et al. (2006) Nature 439:344). As a control, we carried out a parallel selection regime to isolate BFA resistant viruses, which optimally requires the accumulation of two amino acid substitutions in viral proteins (FIG. 4A) (Crotty et al. (2004) supra). Importantly, the selection of BFA resistant variants was carried out at a BFA concentration that initially inhibited viral replication to a similar degree as GA (compare GA and BFA inhibition on the untreated viral population, FIG. 4A). This ensured a similar selective pressure in both drug selection procedures. Following 10 passages in the presence of the inhibitors the sensitivity of each virus to BFA and GA was examined. Strikingly, while the virus grown in BFA had become significantly BFA-resistant, no GA resistance was detected for the virus grown in the presence of the Hsp90 inhibitor (FIG. 4A). To extend this result, we next carried out an independent selection for GA-escape mutants for 20 passages in the presence of inhibitor, representing over 50 replication cycles (FIG. 4B). Strikingly, no resistance to GA was observed under these conditions (FIG. 4B). Conservative theoretical considerations indicate that each passage in the presence of GA should generate at least 2.7×10⁷ potential mutation events in P1 (Drake (1999) Ann. N.Y. Acad. Sci. 870:100), and that mutations offering even a 12% fitness advantage to growth in GA would suffice to completely dominate the viral population under our experimental conditions. Given that the Hsp90 requirement of P1 folding cannot be circumvented by compensatory mutations even after so many generations, it appears that the protein folding pathway of P1 is under strong evolutionary constraints, which limit its capacity to change its sequence without affecting the viability of the virus.

FIG. 4. Poliovirus cannot bypass the Hsp90 requirement. (A) Poliovirus can gain resistance to BFA but not GA within 10 passages. For each passage, 10⁶ viruses (multiplicity of infection (MOI) <0.2) were used to inoculate a new dish in the presence of BFA, GA, or no drug. After 10 passages, the sensitivity of each virus to BFA or GA was tested as in FIG. 1B. Data represents the mean number of PFU/cell and SEM. (B) Poliovirus remains GA-sensitive following extensive serial passage in the presence of GA. For each passage, an MOI of 0.1 to 0.01 was used to inoculate a new dish of cells in the presence GA. ** p<0.01 relative to the virus passaged untreated by t-test.

### Hsp90 Inhibitors Impair Poliovirus Replication in Infected Animals

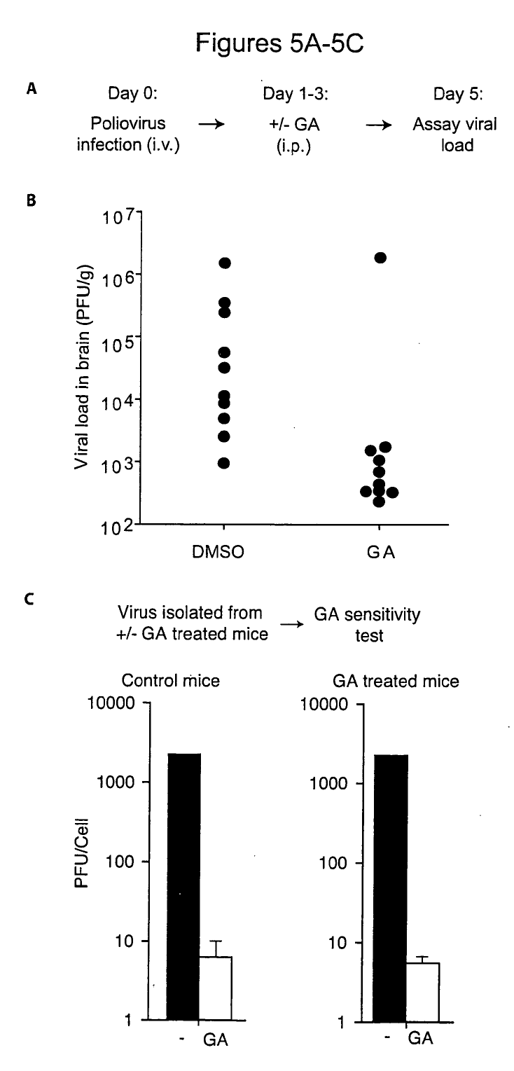

The inability of poliovirus to become Hsp90-independent suggests that protein folding inhibitors may provide an antiviral strategy that can function in vivo without eliciting drug resistance. Despite their use in clinical trials for cancer treatment, the ability of Hsp90 inhibitors to reduce viral replication in infected animals has not been addressed (Dai and Whitesell (2005) Future Oncol. 1:529). It was tested whether Hsp90 inhibitors can impair poliovirus replication in infected mice. It was initially examined whether GA can inhibit the replication of poliovirus in a transgenic mouse model of poliomyelitis extensively used to study the pathogenesis of poliovirus (Crotty et al. (2002) supra; and Vignuzzi et al. (2006) supra). Beginning on the day of infection, the Hsp90 inhibitor GA was administered systemically for four days using a dose and formulation previously shown to inhibit an Hsp90 dependent process in mice (FIG. 5A) (Bucci et al. (2000) Br. J. Pharmacol. 131:13). GA treatment significantly reduced the viral load in the central nervous system (CNS) of poliovirus-infected mice compared to vehicle treated mice in two independent experiments (FIG. 5B).

Since the infected animals may provide several alternative microenvironments for viral evolution, we examined whether the viral population recovered from GA-treated animals five days post infection had acquired drug-resistance (FIG. 5C). Notably, viruses isolated from control and GA treated animals were equally sensitive to the inhibitor; thus, no drug resistance arose in infected animals during GA treatment (FIG. 5C).

FIG. 5. GA inhibits viral replication in poliovirus-infected animals without eliciting drug resistance (A) Outline of the experiment. (B) Viral load in the brains of poliovirus infected cPVR transgenic mice treated with vehicle or GA is expressed as number of PFU per gram of brain (n=10 per group, p<0.01 by Wilcoxon two sample test). (C) Viral populations recovered from GA-treated animals remain GA-sensitive. Poliovirus isolated from the brains of infected animals from FIG. 5B was used to infect HeLa S3 cells at a low multiplicity of infection (MOI; 10⁻⁴) in the presence or absence of 1 μM GA. Virus production was measured after 48 hours by standard plaque assay. Data represents the average number of PFU produced per cell from all ten GA treated animals and four control animals.

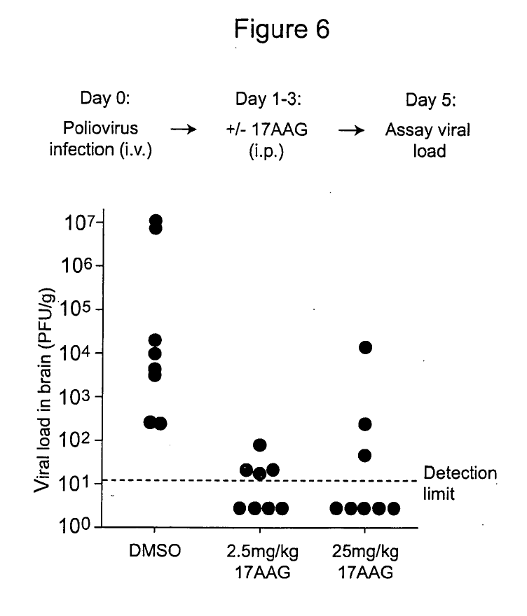

It was next examined the antiviral activity of a first generation GA derivate, 17AAG, which is better tolerated than GA, more effective in crossing the blood-brain barrier and is currently in clinical trials for cancer treatment (Dai and Whitesell (2005) supra; and Waza et al. (2005) Nat. Med. 11:1088). While poliovirus was readily detected in all vehicle treated animals, daily 17AAG treatment dramatically reduced the viral load in the CNS (FIG. 6). In fact, the virus decreased to undetectable levels in 4 of 8 mice receiving a lower 17AAG dose and in 5 of 8 mice receiving a higher dose (FIG. 6). Importantly, the short course of 17AAG treatment did not result in any apparent toxicity to the treated animal. The improved pharmacological properties of 17AAG over GA may account for its dramatic ability to reduce the viral load in the CNS of infected animals even at the lower dose used here. Together, these results provide a proof-of-principle for the hypothesis that inhibitors of chaperone function can effectively block viral replication in infected animals.

FIG. 6. 17AAG inhibits viral replication in poliovirus-infected animals. Viral load in the brains of poliovirus infected cPVR transgenic mice treated with vehicle or 17AAG expressed as in FIG. 5B (n=8 per group, p<0.001 for 2.5 mg/kg group and p<0.005 for 25 mg/kg group by Wilcoxon two sample test). Animals with no detectable virus (4 of 8 mice treated with 2.5 mg/kg 17AAG and 5 of 8 mice treated with 25 mg/kg 17AAG) are plotted below the hatched line indicating the detection limit.

### Example 2

### Hsp90 Inhibitors as Antiviral Agents for the Treatment of Flavivirus Infection

Part 1. Reduction of Viral Replication in Cultured Cells:

Flaviviruses (Includes Hepatitis C Virus, West Nile Virus, Yellow Fever Virus, Dengue Virus):

Standard cultured cells (e.g., Vero or BHK 21) are infected with yellow fever vaccine strain 17D or a laboratory strain of Dengue virus at a multiplicity of infection of 1-5. Hsp90 inhibitors (such as geldanamycin or its derivative 17-AAG) are added post infection. Effects on viral replication are measured at different times after infection, including 24 and 48 hours. Virus production in tissue culture supernatant is measured by standard protocol of plaque assay or 50% tissue culture infective dose tissue culture cells (ie BHK-21). Reduced virus production in the presence of Hsp90 inhibitors is indicative of an antiviral effect mediated by Hsp90 inhibition.

Part 2: Reduction of Viral Replication In Vivo

For any virus family from part 1 in which an in vitro antiviral effect is observed, an animal model for one virus member of the family is tested for antiviral effects in vivo.

Examples of In Vivo Models for Flaviviruses:

Yellow Fever (Vaccine Strain 17D):

Mice (such as C57BL/6) are infected systemically by intravenous or intraperitoneal injection. Mice are systemically treated with Hsp90 inhibitors (such as 17AAG) on the day of infection and every day after infection. A pre-infection dose may be administered. Several tissues (such as pancreas, liver, spleen, brain) are removed at around day four after infection and examined for viral load. Reduced viral load in a tissue from Hsp90 inhibitor treated animals compared to vehicle treated animals indicates an antiviral effect of Hsp90 inhibitors.

If no virus is detectable by systemic infection with yellow fever, an intracranial route of infection is performed. Under these conditions, Hsp90 inhibitors are co-administered intra-cranially with infection. Viral load is then measured in the brains of infected animals between days 2-4.

Dengue Model:

Mice susceptible to systemic infection by dengue virus (interferon knock out mice such as A129, AG129, other interferon receptor knock out mice) are infected by iv or ip route with dengue virus. Animals are treated with Hsp90 inhibitors on the day of infection and on subsequent days by ip administration. Viral load is examined in tissues such as spleen, liver, brain, between days 3-5.

Part 3: Viral Drug Resistance:

The ability of viruses which are susceptible to Hsp90 inhibitors to gain resistance to

Hsp90 inhibitors after serial passage in the presence of Hsp90 inhibitors is tested. A representative virus from each family in part 1 is subjected to serial passage in the presence of Hsp90 inhibitors. Cells are infected at a low MOI (MOI<0.1) and treated with Hsp90 inhibitors. After cytopathic effect (CPE) is observed, the virus is tittered and used to re-infect a new dish of cells in the presence of the Hsp90 inhibitor. This procedure is repeated between 5-10 times. A similar susceptibility to Hsp90 inhibitors after several passages in the presence of the drug compared to the unpassaged virus indicates no drug resistance has arisen.

### Example 3

### Hsp90 Inhibitors as Antiviral Agents for the Treatment of Influenza Virus Infection

Part 1: Inhibition of Viral Replication In Vitro

Experiments are carried out in a manner similar to those described in Example 2, using standard cultured cells (e.g., MDCK). The influenza A strain WSN/33 is used. Additionally, virus production is measured at earlier time points (e.g., 8, 12, 24 hours).

Part 2: Inhibition of Viral Replication In Vivo

Where an in vitro antiviral effect is observed in Part 1, an animal model for one virus member of the family is tested for antiviral effects in vivo, e.g., in an animal model of influenza virus infection.

Influenza Model:

The influenza A virus WSN/33 or another influenza virus is used to infect mice (C57BL/6 or Balb/C) intranasally. Mice are treated with Hsp90 inhibitors intraperitoneally the day of infection and on subsequent days after infection. Between days 3-5 post-infection, the lungs are removed and the viral load examined by plaque assay. A reduction in the viral load of Hsp90 treated animals relative to vehicle treated animals is indicative of an antiviral effect by Hsp90 inhibitors.

Part 3: Viral Drug Resistance:

The ability of viruses which are susceptible to Hsp90 inhibitors to gain resistance to Hsp90 inhibitors after serial passage in the presence of Hsp90 inhibitors is tested. A representative virus from each family in part 1 is subjected to serial passage in the presence of Hsp90 inhibitors. Cells are infected at a low MOI (MOI<0.1) and treated with Hsp90 inhibitors. After cytopathic effect (CPE) is observed, the virus is tittered and used to re-infect a new dish of cells in the presence of the Hsp90 inhibitor. This procedure is repeated between 5-10 times. A similar susceptibility to Hsp90 inhibitors after several passages in the presence of the drug compared to the unpassaged virus indicates no drug resistance has arisen.

### Example 4

### Combination Treatment with Hsp90 Inhibitor and HDAC Inhibitor

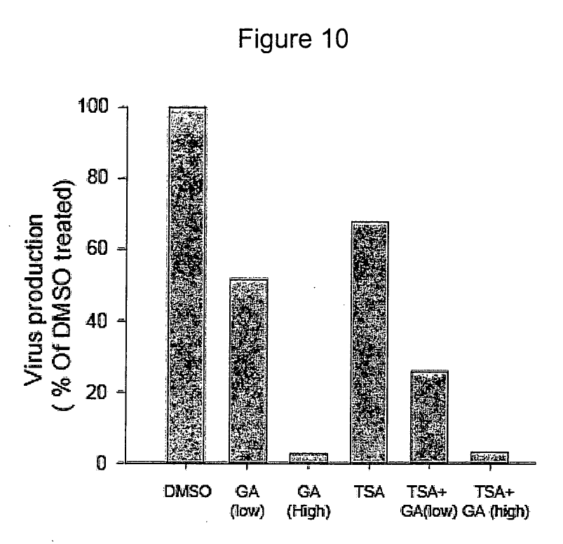

HeLa S3 cells were pretreated with 5 μM trichostatin A (TSA) for 2 hours. Cells were then washed and infected with poliovirus at an MOI of 5 for 15 minutes. Cells were then washed again and geldanamycin (GA) at 0 μM (“DMSO”), 0.06 μM (“low”), or 0.25 μM (“high”) was added with either 0 μM or 5 μM TSA. Virus production was measured after 7 hours by standard plaque assay. The results are shown in FIG. 10.

The effects of HDAC inhibition on picornavirus replication were examined in cultured cells. Cells treated with the HDAC inhibitor trichostatin A (TSA) showed a maximal reduction in virus production of 30% relative to DMSO treated controls (FIG. 10). When HDAC inhibition was combined with sub-saturating Hsp90 inhibition (GA low, ˜50% reduction in virus production), a cumulative effect was observed whereby virus production was reduced by >70% of DMSO treated controls (see TSA+GA (low) condition). When saturating amounts of Hsp90 inhibitors were used in combination with TSA, no further inhibition was observed beyond that achieved by Hsp90 inhibition alone (compare GA (high) with TSA+GA (high)). Thus, Hsp90 appears to be acting downstream of HDAC inhibition.

### Example 5

### Effect of Hsp90 Inhibitors on RSV Production in Cultured Cells

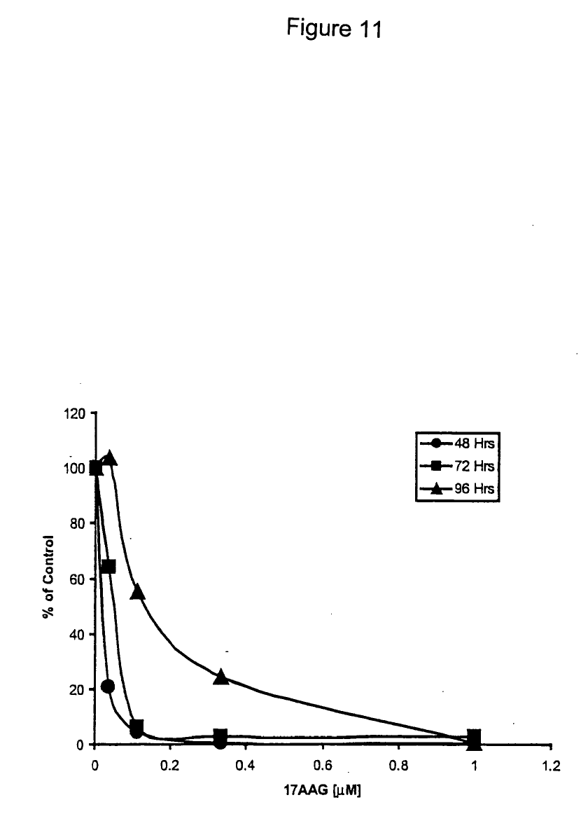

The effect of Hsp90 inhibitors on Respiratory Syncytial virus (RSV) production in cultured cells was examined. Hep-2 cells were infected in vitro with RSV-A2 at a low multiplicity of infection in the presence of different concentrations of the Hsp90 inhibitor 17AAG. The data are shown in FIG. 11. At the indicated time points, aliquots of the media were removed and the amount of virus in the supernatant quantified by end point titration. The data represent the percent of virus production relative to DMSO treated control.

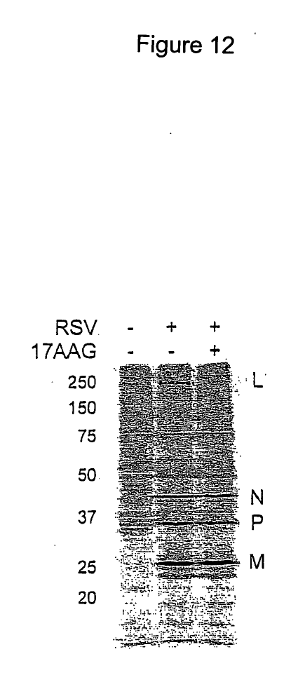

Hep-2 cells were infected with RSV, strain A2. After 16 hours, the cells were incubated in the presence of Actinomycin D (1 μM) for 2 hours. The media was then replaced with methionine and cysteine free media with Actinomycin D and either 17AAG (5 μM) or DMSO as a control. After 1 hour, the cells were pulsed with radioactive methionine and cysteine for 2 hours, lysed and viral protein immunoprecipitated with a goat anti-RSV antibody. Immunoprecipitated proteins were analyzed by 12% SDS-PAGE and autoradiography. The data are shown in FIG. 12. The molecular weights and the viral proteins are indicated. As shown in FIG. 12, Hsp90 inhibition causes degradation of L protein, the Respiratory Syncytial virus polymerase.

### Example 6

### Effect of Hsp90 Inhibitors on Influenza A Replication in Cultured Cells

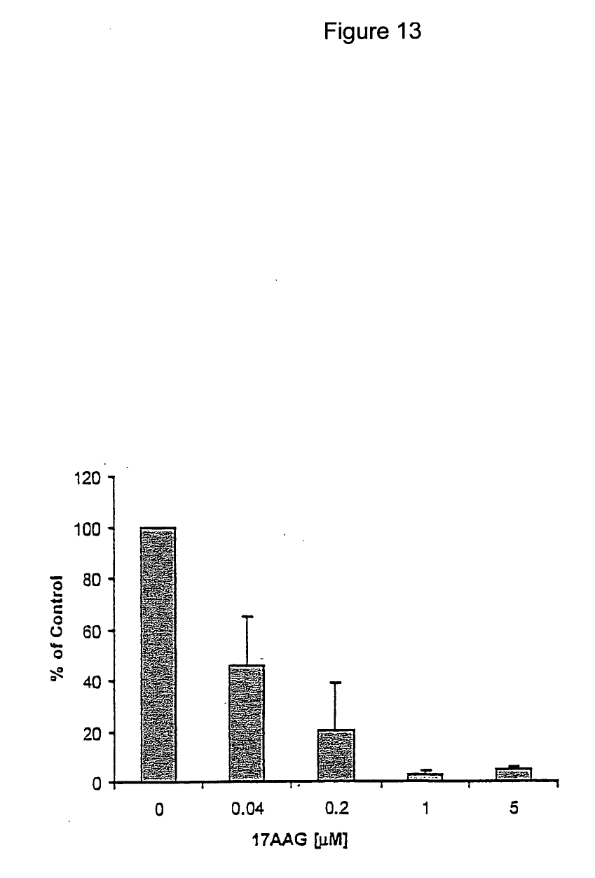

The effect of Hsp90 inhibitors on Influenza A replication in cultured cells was examined. MDCK cells were infected at a low multiplicity of infection with Influenza A H1N1, strain PR/8, in the presence of different concentration of the Hsp90 inhibitor 17AAG. Virus production was measured by end point titration after 24 hours. The data, presented in FIG. 13, represent the percent of virus production relative to DMSO treated control.

### Example 7

### Effect of Hsp90 Inhibitors on Yellow Fever Virus Replication in Cultured Cells

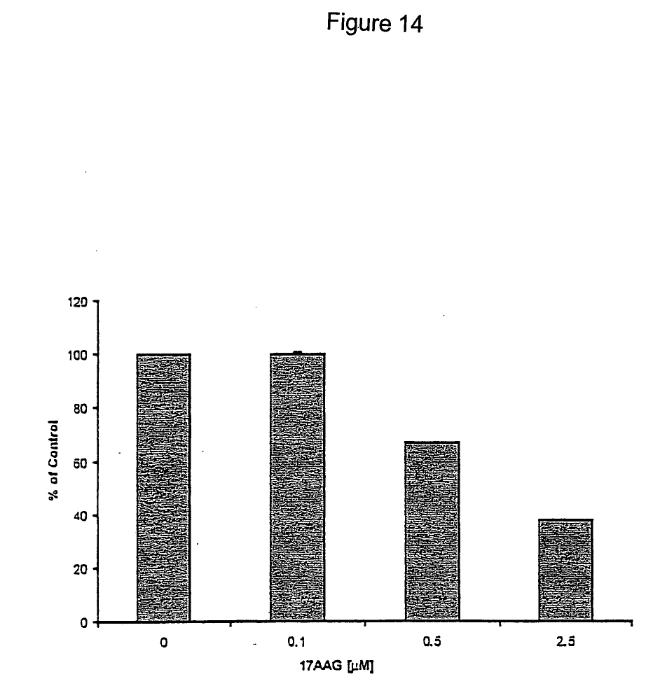

The effect of Hsp90 inhibitors on Yellow Fever Virus replication in cultured cells was examined. BHK21 cells were infected at an multiplicity of infection of one with YFV strain 17D in the presence of different concentration of the Hsp90 inhibitor 17AAG. Virus production was measured by plaque assay after 48 hours. The data, presented in FIG. 14, represent the percent of virus production relative to DMSO treated control.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating an RNA virus infection in an individual, the method comprising administering to an individual in need thereof an agent that reduces the activity of a host cell protein that is required for maturation of one or more proteins encoded by the RNA virus, wherein variants of the RNA virus that are resistant to the agent are not produced in detectable amounts for at least 2 days following beginning of administration of the agent.

2. The method of claim 1, wherein the agent that reduces the activity of a host cell protein that is required for maturation of one or more proteins encoded by the RNA virus is a heat shock protein inhibitor.

3. The method of claim 2, wherein the agent is a benzoquinone inhibitor of Hsp90.

4. The method of claim 3, wherein the benzoquinone is a geldanamycin derivative.

5. The method of claim 4, wherein the geldanamycin derivative is 17-allylamino-17-demethoxygeldanamycin, 17-(dimethylaminoethylamino)-17-demethoxygeldanamycin, 17-[2-(Pyrrolidin-1-yl)ethyl]amino-17-demethoxygeldanamycin, or 17-(Dimethylaminopropylamino)-17-demethoxygeldanamycin.

6. The method of claim 2, wherein the agent is a benzenediol inhibitor of Hsp90.

7. The method of claim 5, wherein the agent is radicicol, or a radicicol derivative.

8. The method of claim 1, wherein variant virus that is resistant to the agent is not produced in detectable amounts for at least 3 days following beginning of administration of the agent.

9. The method of claim 1, further comprising administering a second agent that reduces the activity of a host cell protein that is required for maturation of one or more proteins encoded by the RNA virus.

10. The method of claim 9, wherein the second agent is a histone deacetylase (HDAC) inhibitor.

11. The method of claim 10, wherein the HDAC inhibitor is an HDAC6 inhibitor.

12. The method of claim 10, wherein the HDAC inhibitor is suberoylanilide hydroxamic acid.

13. The method of claim 1, wherein the RNA virus infection is a picornavirus infection.

14. The method of claim 13, wherein the picornavirus is a poliovirus, a rhinovirus, or a coxsackievirus.

15. The method of claim 1, wherein the RNA virus infection is a flavivirus infection.

16. The method of claim 15, wherein the flavivirus is West Nile Virus, Hepatitis C Virus, Yellow Fever Virus, or Dengue Virus.

17. The method of claim 1, wherein the RNA virus infection is an influenza virus infection.

18. The method of claim 1, wherein the RNA virus infection is a paramyxovirus infection.

19. The method of 18, wherein the paramyxovirus is respiratory syncytial virus.

20. The method of claim 1, wherein the individual is treatment naïve.

21. The method of claim 1, wherein the individual is a treatment failure patient.

22. The method of claim 21, wherein the individual was previously treated with an anti-viral agent other than an agent that reduces the activity of a host cell protein that is required for maturation of one or more proteins encoded by the RNA virus, and who developed resistance to the anti-viral agent.


SEQUENCE LISTING (1 sequence)

SEQ ID NO: 1 - protein, 724 aa, Homo sapiens
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD